(19) United States
(12) Patent Application Publication
(10) Pub. No.: US 2026/0167928 A1
(43) Pub. Date: Jun. 18, 2026

(54) INDUCED PROTEINOPATHY MODELS

(71) Applicant: The Brigham and Women's Hospital, Inc., Boston, MA (US)

(72) Inventors: Vikram Khurana, Brookline, MA (US); Isabel Lam, Cambridge, MA (US); Alain Ndayisaba, Boston, MA (US); Jackson Sandoe, Quincy, MA (US)

(21) Appl. No.: 18/711,400

(22) PCT Filed: Nov. 18, 2022
(86) PCT No.: PCT/US2022/050468

§ 371 Date: May 17, 2024

Related U.S. Application Data
(60) Provisional application No. 63/264,275, filed on Nov. 18, 2021.

(51) Int. Cl. C12N 5/079; C07K 14/47; C12N 5/0793; C12N 5/09; C12N 9/22; C12N 15/11; C12N 15/85; G01N 33/50

(52) U.S. Cl. CPC C12N 5/0622; C07K 14/4702; C07K 14/4711; C12N 5/0619; C12N 5/0693; C12N 9/226; C12N 15/111; C12N 15/85; G01N 33/5023; C07K 2319/60; C12N 2310/20; C12N 2503/02; C12N 2506/45; C12N 2510/00; C12N 2830/003; C12N 2840/203; G01N 2500/10

(57) ABSTRACT

Disclosed herein are methods and compositions useful in screening a compound for its effect on proteotoxicity and proteinaceous inclusions involved in neurological disease.

### CLAIM OF PRIORITY

This application claims the benefit of U.S. Provisional Patent Application Ser. No. 63/264,275, filed on Nov. 18, 2021. The entire contents of the foregoing are hereby incorporated by reference.

### TECHNICAL FIELD

This invention relates to making and using cell models of neurologic disease in which toxic proteinaceous inclusions (of proteins such as alpha-synuclein (α-syn), tau or TDP-43) are rapidly induced.

### BACKGROUND

Neurodegenerative diseases devastate individuals and societies and pose an ever-increasing public health threat. The most common neurodegenerative diseases are associated with misfolding and aggregation of distinct proteins in neurons and glial cells. The characteristic aggregates are amyloids rich in beta-sheet secondary structure. For instance, the classic “beta-amyloidopathy” is Alzheimer's disease (AD), associated with extracellular deposition of beta-amyloid plaques (1). AD is also a “tauopathy” because it is associated with aggregation of the microtubule-associated protein tau. Other tauopathies include frontotemporal dementias (FTDs), corticobasal ganglionic degeneration (CBGD) and progressive supranuclear palsy (PSP). A plethora of proteinaceous aggregates with distinct biochemical and ultrastructural properties form in neurons and glia in these diseases, including the classic intraneuronal neurofibrillary tangles (NFTs) in Alzheimer's disease (2). “TDP-43opathies” include amyotrophic lateral sclerosis (ALS) and certain frontotemporal dementias (FTDs) and are associated with the nucleocytoplasmic relocalization and aggregation of TDP-43. “Synucleinopathies” include multiple system atrophy (MSA), dementia with Lewy bodies (DLB) and Parkinson's disease (PD). These are the diseases associated with the aggregation of the protein alpha-synuclein (α-syn), a small 14-kDa protein associated with phospholipids in membranes and synaptic vesicles in neurons (3).

These misfolding-prone proteins have been causally linked to neurodegeneration principally because locus multiplication or point mutations in the genes encoding these proteins cause familial forms of these neurodegenerative diseases transmitted in an autosomal dominant fashion. Recently, beyond the accumulation of the toxic protein itself, it is becoming increasingly clear that key aspects of the disease are also encoded in distinct conformations of these proteins. Distinct conformers of amyloid proteins—that borrow the name “strains” from the prion field—can be isolated from patients with distinct diseases that arise from the misfolding of the same protein. Just as with prions, inoculation of distinct strains into the brains of mice leads to distinct diseases. Moreover, within the brain of a single patient, multiple morphological conformers can be seen, correspondingly diverse at the ultrastructural level. While neuropathologic examination of these inclusions has been tremendously helpful, it is also clear that biological insights are sorely limited. Antibodies, for example to phosphorylated α-syn or tau, indiscriminately label diverse inclusions. In some regions of the brain, there is association with neurodegeneration (that is, frank neuronal loss) but in other regions there is no association with neuronal loss. It is plausible that a deeper understanding of these inclusions would shed light on these diverse biological outcomes of protein aggregation. Moreover, modelling distinct inclusions in a native human cellular context may have implications for diagnostics—currently there is a major need for development of radiotracers that identify these pathologic lesions to stratify patients for disease-modifying therapies. The drug aducanumab, recently approved by the FDA, only achieved modest success in clinical trial by virtue of application specifically to patients with beta-amyloid deposition identified by PET (4). Currently, there is no tracer for α-syn and only limited choices for tau aggregation. These concepts have been extensively reviewed recently (5).

### SUMMARY

Provided herein are PiggyBac vectors comprising one or more, preferably all, of the following: a sequence encoding a target protein selected from the group consisting of TAR DNA-binding protein (TARDBP, TDP-43), apolipoprotein E (ApoE), α-synuclein (SNCA), beta-amyloid, amyloid precursor protein (APP), and tau, wherein the target protein causes formation of proteotoxic or proteinaceous inclusions in the cell; at least one pair of insulators; at least one antibiotic selection gene; an inducible promoter, e.g., tet-inducible promoter; optionally, a neuronal differentiation transcription factor (e.g., Ngn2, NFIB, or Sox9) gene; and a herpes simplex virus thymidine kinase selection gene.

Also provided herein are methods of generating a human transgenic cellular model of neurodegenerative proteinopathies comprising: transducing a human cell with a PiggyBac vector, wherein the PiggyBac vector comprises a sequence encoding a target protein selected from the from the group consisting of apolipoprotein E (ApoE), TARDBP, α-syn, beta-amyloid, amyloid precursor protein (APP), and tau, wherein expression of the target protein causes proteotoxic or proteinaceous inclusions in the cell, wherein the PiggyBac vector comprises at least one pair of insulators, at least one antibiotic selection gene, and a herpes simplex virus thymidine kinase selection gene.

In some embodiments, the target protein is α-syn or TARDBP. In some embodiments, the sequence encoding the target protein encodes an amino acid sequence comprising a wild type version of the target protein, or an amino acid sequence containing a disease risk-associated polymorphism or mutation. In some embodiments, the alpha-synuclein comprises E35K, E46K, and/or E61K point mutations, or the TARDBP comprises Q331K or M337V point mutations. In some embodiments, a green fluorescent protein (GFP) is linked to the target protein.

In some embodiments, the PiggyBac vector comprises an inducible promoter, preferably a tet-inducible promoter. In some embodiments, the PiggyBac vector comprises 2 or 4 insulators, preferably UCOE insulator, iA4 insulator, cHS4 insulator, or iA2 insulator.

In some embodiments, the human cell is selected from the group consisting of a pluripotent stem cell (iPSC), an embryonic stem cell (ESc), and a cell from an immortalized cell line. In some embodiments, the human cell is the iPSC.

In some embodiments, the PiggyBac vector further comprises a neuronal differentiation transcription factor (e.g., Ngn2, NFIB, or Sox9) coding sequence. In some embodiments, the iPSC is differentiated to a cortical neuron cell by expression of Ngn2; to an astrocyte by expression of NFIB; or an oligodendrocyte by expression of Sox9.

In some embodiments, the iPSC comprises a disease risk-associated polymorphism or mutation in a gene selected from a group comprising α-syn, TARDBP, APP, tau or ApoE.

Also provided herein are methods comprising generating a human cell comprising a target gene, wherein the target gene is introduced into the genome of the cell by CRISPR, encoding a target protein selected from the group consisting of apolipoprotein E (ApoE), TARDBP, α-syn, beta-amyloid, amyloid precursor protein (APP), and tau, wherein the target protein causes formation of proteotoxic or proteinaceous inclusions in the cell and is introduced into the AAVS1 locus or STMN2 locus.

In some embodiments, the target gene is introduced into the STMN2 locus. In some embodiments, the methods further include introducing an Ngn2 gene and a tet-inducible promoter.

In some embodiments, the target protein comprises an amino acid sequence containing a disease risk-associated polymorphism or mutation.

In some embodiments, the target protein is α-syn or TARDBP. In some embodiments, the α-syn comprises E35K, E46K, and E61K point mutations, or the TARDBP comprises Q331K or M337V point mutations. In some embodiments, a fluorescent protein, e.g., green fluorescent protein (GFP), is linked to the target protein. In some embodiments, the human cell is selected from the group consisting of pluripotent stem cell (iPSc), an embryonic stem cell (ESc), and a cell from an immortalized cell line, preferably, an U2OS cell.

Also provided herein are isolated human cells comprising a PiggyBac vector, wherein the PiggyBac vector comprises a sequence encoding a target protein selected from the group consisting of apolipoprotein E (ApoE), α-syn, beta-amyloid, amyloid precursor protein (APP), and tau, wherein expression of the target protein causes proteotoxic or proteinaceous inclusions in the cell, wherein the PiggyBac vector comprises at least one pair of insulators, at least one antibiotic selection gene, and a herpes simplex virus thymidine kinase selection gene.

In some embodiments, the isolated human cell is selected from the group consisting of pluripotent stem cell (iPSc), an embryonic stem cell (ESc), and a cell from an immortalized cell line, preferably an U2OS cell. In some embodiments, the human cell is differentiated into neurons or glial cells, preferably cortical neurons, dopaminergic neurons, astrocytes, oligodendrocytes, microglia.

Additionally, provided herein are isolated human cells comprising an apolipoprotein E (ApoE), α-syn, TARDBP, beta-amyloid, amyloid precursor protein (APP), or tau gene expressing from an AAVS1 locus and an Ngn2 gene, wherein the cell is generated by: contacting a human cell, preferably an hESc or iPSC cell, with an RNA-guided Cas9 nuclease and gRNA directed to a sequence in the AAVS1 locus, and a sequence under an inducible promoter encoding ApoE, TARDBP, α-syn, beta-amyloid, APP, or tau, under conditions allowing insertion of the ApoE, TARDBP, α-syn, beta-amyloid, APP, or tau gene into the AAVS1 locus; differentiating the human cell into a neuron or glial cells by expressing Ngn2; and maintaining the cell under conditions suitable for expression of ApoE, TARDBP, α-syn, beta-amyloid, APP, or tau to cause proteotoxic or proteinaceous inclusions in the cell.

Further, provided herein are isolated human cells comprising (i) a sequence encoding apolipoprotein E (ApoE), TARDBP, α-syn, beta-amyloid, amyloid precursor protein (APP), or tau protein inserted in a STMN2 or AAVS1 locus, and (ii) an exogenous Ngn2 gene, wherein the cell is generated by: contacting the human cell, preferably an hESc or iPSC cell, with an RNA-guided Cas9 nuclease and gRNA directed to a sequence in the AAVS1 or STMN2 locus, and a sequence encoding TARDBP, ApoE, alpha-synuclein, beta-amyloid, APP, or tau, under conditions allowing insertion of the TARDBP, ApoE, α-syn, beta-amyloid, APP, or tau gene into the AAVS1 or STMN2 locus; differentiating the human cell into a neuron or glial cells by expressing the Ngn2; and optionally maintaining the differentiated cells under conditions suitable for expression of TARDBP, ApoE, α-syn, beta-amyloid, App, or tau to cause proteotoxic or proteinaceous inclusions in the cell.

Additionally, provided herein are methods of identifying a candidate compound for treating a neurodegenerative condition associated with proteotoxic or proteinaceous inclusions in neuronal or glial cells. The methods comprise: contacting a human cell as described herein (e.g., generated using a method or composition as described herein) with a test compound, optionally in the presence and absence of fibrils; evaluating a level of proteotoxic or proteinaceous inclusions in the human cell in the presence and absence of the candidate compound; and selecting as a candidate compound a test compound that reduces the level of proteotoxic or proteinaceous inclusions in the human cell in the presence of fibrils.

Further, provided herein are methods for identifying a candidate gene therapy for treating a neurodegenerative condition associated with proteotoxic or proteinaceous inclusions in neuronal or glial cells. The methods comprise: contacting a human cell as described herein (e.g., generated using a method or composition as described herein) with a vector comprising a single gene or library of genes that over-express, knockdown or knock-out one or more genes in the human genome; evaluating a level of proteotoxic or proteinaceous inclusions in the human cell in the presence and absence of the candidate compared; and selecting as a candidate gene a specific gene target or combination or targets that reduces the level of proteotoxic or proteinaceous inclusions in the human cell.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. For example, proof-of-principle is demonstrated most of all in this application for synucleinopathies. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D. Exemplary PiggyBac expression vectors. 1A-C. three cartoons showing elements of exemplary modified vectors, before transgene insertion, or with an NGN2 or NGN2-α-syn insertion, respectively. 1B. PlasmidID #1021, a non-neuronal expression construct for high-throughput screening purposes is an example used to generate mutant α-syn-A53T (or the control α-syn-A53T missing the pro-aggregation NAC domain “A53T-ΔNAC”). An mKate2 sequence in this example was used after the IRES sequence to label the cells that have integrated the vector with a red fluorescent protein. 1C. exemplary PlasmidID #1022, a neuronal construct with NGN2-2A-Puro insert, allowing for simultaneous Ngn2 expression (to transdifferentiate pluripotent stem cells into cortical neurons) and second antibiotic (puromycin) selection. A SNAP-TAG (New England Biolabs), a protein tag that forms a highly stable, covalent thioether bond with fluorophores or other substituted groups when appended to benzylguanine, was used in this exemplary plasmid after the IRES sequence to label the cells that have integrated the vector. This enables a large selection of substrates for fluorescent labeling of cells for immunofluorescence or FACS sorting. Constructs for transdifferentiation to astrocytes (NFIB) (6) and oligodendrocytes (SOX9) (7) have also been generated with the same backbone. 1D. a linearized schematic of the plasmid #1022 depicted in FIG. 1C.

FIG. 2. Overview of induced proteinopathy models in this patent application, with application to alpha-synucleinopathy. PiiN: PiggyBac induced inclusion neurons. In these models a PiggyBac-NGN2 construct (identical or similar to FIG. 1C/D) is coupled with α-syn overexpression. This enables rapid, scalable transdifferentiation of iPSC to neurons. α-syn overexpression is achieved in one of two ways. First, α-syn can be physiologically over-expressed. In these lines, PiggyBac-NGN2 is introduced into iPSC from patients with Parkinson's disease/dementia caused by increased copy number (e.g., triplication) of wild-type α-syn, or familial mutation in SNCA (e.g. A53T), and their mutation-corrected controls (e.g. copy number knockdown controls SNCA 2-copy, SNCA 0-copy, or A53T-corrected “CORR” line). Second, α-syn can be over-expressed through transgenic over-expression. The transgene is introduced in one of three ways: PiggyBac random integration, targeting to a lineage-specific locus (e.g., STMN2 for pan-neuronal expression), or targeting to a safe harbor locus (e.g. AAVS1).

FIGS. 3A-D. PiiN Triplication Model (“Seeded” physiologic overexpression). 3A. Physiologic α-syn overexpression model seeded with synthetic and brain-derived pre-formed fibrils (PFFs). Schematic outlining the generation of human neurons with differential SNCA copy numbers starting from PD patient fibroblasts with SNCA locus triplication. This is an example of a series of iPSC lines into which the Ngn2 containing transgene (FIG. 1C-D) was introduced to facilitate trans-differentiation into cortical neurons. In detail, fibroblasts from a PD patient (Iowa kindred) harboring triplication of SNCA locus were differentiated to iPSCs by introduction of Yamanaka factors. At the iPSC stage, SNCA copy number was lowered by CRISPR/Cas9 editing to 2 copies (reduced copy, i.e. wild-type levels) and complete knockout (0-copy). After generation of the isogenic lines differing in α-syn copy number, NGN2 gene along with the TET-ON system was introduced to each series by an all-in-one PiggyBac vector mediated transposition (See FIG. 1C-D). The cells were trans-differentiated to cortical neurons by doxycycline induced expression of Ngn2 (induced neurons; iNs). The cells were aged for at least three weeks (21 days in vitro; 21 DIV) or more. 3B. Exposure of 4-copy, 2-copy and 0-copy α-syn isogenic transdifferentiated neurons to synthetic pre-formed α-syn fibrils (10 μg/mL). Pathologic α-syn (indicated by phosphorylation of Ser129; Abcam Ab51253); was observed in 4-copy>>2-copy lines but not in the 0-copy lines. Neuronal processes and nuclei were detected using an antibody against BIII Tubulin (Biolegend 801201) and Hoechst 33342 DNA stain (Invitrogen). Z-stacks were taken on a Nikon TiE confocal microscopy system. 3C. An exemplary schematic illustration of protein misfolding cyclic amplification assay that was used to capture and amplify α-syn fibrils from postmortem brain. 3D. Diverse inclusions visible after seeding PiiN induced inclusion neurons with brain-derived PFFs (left) are reminiscent of intraneuronal α-syn inclusions seen in human brain (right). PFFs generated from cyclic amplification (FIG. 3C) of multiple system atrophy (MSA) and Parkinson's disease (PD) patient brain-derived α-syn. Sarkosyl-insoluble fraction of brain lysates derived from an MSA and PD case was co-incubated with synthetic monomeric α-syn. After multiple rounds of amplification, quantification and sonication of resultant fibrils, the PFFs were introduced into iPSC-derived transdifferentiated neurons harboring triplication of SNCA (4-copy). Pathologic α-syn is detected by staining for phosphorylated α-syn at Ser129 (Abcam Ab51253).

FIGS. 4A-F. Comparing Triplication to Tg^{PB} PiiN models. 4A. Schematic showing the generation of a line that over-expresses untagged wild-type α-syn at high levels in a doxycycline-dependent way. The PiggyBac-SNCA-WT-IRES-NGN2 plasmid was stably integrated into a patient A53T mutation-corrected (“CORR”) iPSC line. The CORR iPSC line is derived from patient fibroblast harboring the SNCA A53T mutation (described in Chung et al., Science 2013 (8)). The fibroblast line was reprogrammed using mRNA/microRNA-based reprogramming (Stemgent). The mutation correction (T53 to A53) was performed by Cas9-based genome editing that was plasmid based with an all-in-one vector (px485-GFP) encoding Cas9 and a gRNA directed to SNCA exon 3 that encodes amino acid 53. An asymmetric 127 nt repair donor was introduced that encoded the wild-type SNCA sequence. Transfection was with Lipofectamine 3000 (Invitrogen). Enrichment was with flow cytometry to sort GFP-positive cells. Transgenic iPSCs are trans-differentiated to cortical neurons by doxycycline induced expression of Ngn2, and inclusions in CORR/Tg^{PB-SNCA-WT} neurons subsequently induced by exposure to PFFs. 4B. PiggyBac bicistronic expression vector encoding the toxic protein of interest (in this case untagged wild-type α-syn) as well as Ngn2 under a tetracycline promoter. Separately, the vector encodes the 4^{th} generation reverse tetracycline transactivator and the other features listed in FIG. 1. 4C: Exposure of CORR/Tg^{PB-SNCA-WT} neurons to synthetic WT PFFs and brain-derived PD and MSA PFFs efficiently induces diverse inclusion morphologies within 14 days. Day 11 neurons were seeded with 10 μg/ml pre-sonicated PFFs. Neurons were fixed and immunostained for α-syn phosphorylated at Serine 129, β-III tubulin, and Hoechst nuclear stain at Day 25 of differentiation (FIG. 4C, 4E). Z-stacks were taken on a Nikon TiE confocal microscopy system. 4D: Western blot analysis of α-syn levels in SNCA 0-copy, SNCA 2-copy, SNCA 4-copy neurons, CORR and isogenic CORR/Tg^{PB-SNCA-WT} neurons at Day 25 of neuronal differentiation. Antibodies against total α-syn (MJFR1 ab138501, epitope: 118-123), GAPDH (Sigma G8796) and β-III Tubulin (Biolegend 801201) were used. Western blots were visualized with the LiCor Odyssey CLx. 4E: Quantification of α-syn normalized to GAPDH from western blot shown in FIG. 4D confirms highly increased wild-type α-syn expression via introduction of a PiggyBac wild-type transgene. The graph represents the mean+/−standard deviation of three technical replicates per condition. 4F: Synthetic PFF seeding in CORR/Tg^{PB-SNCA-WT} neurons results in highly increased accumulation of α-syn phosphorylated at Serine 129 (pS129-α-syn, a hallmark feature of Parkinson's disease) compared to SNCA 0-copy, SNCA 2-copy and SNCA 4-copy neurons, based on quantification of pS129 immunostaining. Neurons were exposed to 10 μg/mL pre-sonicated synthetic wild-type PFFs at day 11 of differentiation and incubated for 14 days pre-analysis.

FIGS. 5A-H. sfGFP-tagged lines: Comparing Tg^{PB} to Tg^{STMN2} PiiN models. 5A. To develop a live-cell reporter that specifically reads out α-syn inclusions, we selected an aggregation-prone mutant of α-syn (A53T) and tagged its C-terminal end with sfGFP. As a control, we utilized an A53T construct in which the NAC domain (amino acid residues 60-95) was deleted. 5B. Left: STMN2 expression is considerably higher in neuron than glia (scRNAseq data, human cortex; neuroexpresso.org). Right: Targeting of α-syn expressing transgenes (in this case A53T-sfGFP and A53T-ΔNAC-sfGFP as control) by CRISPR/Cas9 to the STMN2 locus to enable neuron-specific expression of the transgene without use of doxycycline. The system functions with conventional differentiation (C and D) or transdifferentiation (E). 5C. Neurons generated through neural induction according to manufacturer protocols (StemCell Technologies 05835) followed by terminal neural differentiation in neurobasal medium supplemented by BDNF/dbCAMP/GDNF. Analysis at day 10 is shown: GFP signal was immunocolocalized with MAP2 (Abcam AB92434). 5D. Forebrain organoids generated in Khurana lab (→EB→matrigel droplet→bioreactor) from the H9 HESc Stmn2 α-syn (A53T) knock-in line. Shown here are day 30 organoids generated with the STEMdiff Cerebral Organoid Kit according to manufacturer protocols (StemCell Technologies, cat. no. 08570). GFP signal is immunocolocalized with β-III Tubulin (Covance MMS-435P). At day 180 these organoids contained GFAP-positive astrocytes that were predictably GFP-negative (not shown). 5E. A PiggyBac construct (plasmid 1022; see FIG. 1C/D) enables direct conversion from hESc into a nearly pure population of cortical-type neurons expressing α-syn constructs. In this case doxycycline is only used for transdifferentiation and then withdrawn. The figure shows glutamatergic neurons transdifferentiated from the cells with doxycycline-inducible Ngn2. Dox is then withdrawn and α-syn remains expressed. Exposure to synthetic pre-formed α-syn fibrils (PFFs; 10 μg/mL) for two weeks leads to robust seeding and neuritic aggregates in the A53T (right, upper) but not the A53T-ΔNAC (right, lower) condition. The A53T line unexposed to PFFs (left lower) served as a control. 5F. A modified PiggyBac (construct #1018 Khurana lab) expresses Ngn2 and sfGFP-tagged α-syn-A53T (Tg^{PB-A53T-sfGFP}) or the ΔNAC control (Tg^{PB-ΔNAC-sfGFP}). 5G. Western blot analysis of α-syn levels comparing PiggyBac transgene expression to transgene knock-in at the neuron-specific STMN2 locus at day 25 of neuronal differentiation. Antibodies against total α-syn (MJFR1 ab138501, epitope: 118-123), GAPDH (Sigma G8796) and β-III Tubulin (Biolegend 801201) were used. Western blots were visualized with the LiCor Odyssey CLx imaging system. 5H: Quantification of FIG. 5G. PiggyBac transgene expression of α-syn results in significantly higher protein expression compared to α-syn expression under the STMN2 locus. The graphs represent the mean+/−standard deviation of three technical replicates per condition.

FIGS. 6A-H. PiiN-sfGFP Models Reveal Distinct Inclusions Form in Parallel. 6A: Schematic showing the generation of lines that over-express an aggregation-prone mutant of α-syn (A53>T) and are either untagged or tagged at its C-terminal end with sfGFP. As a control, we utilized a construct in which the NAC domain (amino acid residues 60-95) was deleted. 6B: Inclusion formation in PiiN Tg^{PB-A53T} neurons upon A53T-PFF exposure occurs irrespective of protein tagging. Accumulation of α-syn pS129 is not significantly altered by sfGFP tag. Day 11 neurons exposed to 10 μg/mL human recombinant A53T-PFFs were fixed 14 days later and immunostained; antibodies against pS129 (Abcam 51253) and β-III Tubulin (Biolegend 801201) were used, and Hoechst 33342 stain for nuclei labeling. 6C: Sequential Triton X-100/sodium dodecyl sulfate (SDS) extraction of PFF-treated transgenic neurons reveals the presence of insoluble α-syn aggregates. Neurons were PFF-treated on Day 11 (10 μg/mL) and harvested at day 25 of differentiation. Antibodies against α-syn pS129 (Abcam ab51253) and total α-syn (15G7, provided by Dr. Ulf Dettmer) were used. Western blots were visualized using the iBright imaging system. 6D: Schematic outlining different inclusion morphologies occurring in PFF-seeded CORR/Tg^{-PB-A53T-sfGFP} transgenic neurons. The cartoon highlights the three main types of inclusions detected: (a) round cytoplasmic, (b) ribbon-like cytoplasmic inclusions that appear fibrillar, and (c) neuritic inclusions. The image inset shows live CORR/Tg^{PB-A53T-sfGFP}+PFF neurons visualized by sfGFP. 6E: Neuritic and ribbon-like fibrillar inclusions stain for established markers of Lewy pathology including α-syn pS129, ubiquitin and p62, while lipid-rich inclusions stain exclusively for α-syn pS129 but not ubiquitin and p62. Neurons were seeded with PFFs on day 11 and fixed for immunostaining on day 25. Antibodies against ubiquitin (Millipore Sigma, clone FK2), p62 (EMD Millipore, clone 20F1.1) and α-syn pS129 (Abcam, ab51253) were used. Z-stacks were taken on a Nikon TiE confocal microscopy system. 6F: Upper panel: GFP-immunogold labeling and subsequent electron microscopy of CORR/Tg^{PB-A53T-sfGFP}+PFF neurons differentiated for 25 days reveals vesicle and lipid droplet accumulation in lipid-rich inclusions, aberrant neuritic morphology and fibrillar species likely corresponding to the inclusion subtypes observed via immunostaining. Lower panel: Correlative light and electron microscopy of PD postmortem brain (left image from Shahmoradian et al., Nat Neurosci 2019 (9), middle and right image unpublished) reveals inclusion morphologies reminiscent of inclusion subtypes detected in the PiiN model. Left image: white arrowheads-mitochondria, yellow arrowhead-densely compact membranous structure; middle image: yellow arrowheads-thin filaments, blue arrowheads-thick filaments. 6G: Day 25 CORR/Tg^{PB-A53T-sfGFP}+PFF stained with a neutral lipid dye (LipidSpot Lipid Droplet Stain, Biotium 70069) confirm the presence of a lipid-rich inclusion subtype. Hoechst 33432 (Invitrogen) was used as a nuclear label. 6H: Inclusion subtype distribution in CORR/Tg^{PB-A53T-sfGFP}+PFF neuronal cultures from FIG. 6G was assessed by manual counting of 1000 Hoechst positive nuclei across ten image frames (20× objective, Nikon TiE epifluorescence microscope), and total number of inclusions captured within the frame (neuritic inclusions) or within cell body boundaries (round cytoplasmic-, ribbon-like fibrillar inclusion) was assessed.

FIGS. 7A-D. Tg^{PB-E3xK} model can lead to BODIPY+ inclusions. 7A: Schematic showing the generation of lines that over-express an amplification of the familial PD mutation E46K with increased membrane affinity, α-syn-E35K, E46K, E61K (E3xK; CORR/Tg^{PB-E3xK}). The constructs are either untagged or tagged at their C-terminal end with sfGFP. 7B: The E3xK model spontaneously forms cytoplasmic inclusions; here, the inclusions in CORR/Tg^{PE-E3xK-sfGFP} neurons are pS129-positive but stain negatively for Lewy body marker ubiquitin. Labeling of neutral lipids using BODIPY dye reveals that E3xK inclusions are lipid-rich. Antibodies against ubiquitin (Millipore Sigma, clone FK2), pS129 (Abcam ab51253) and LipidSpot Lipid Droplet Stain (Biotium 70069) were used. 7C: Correlative light and electron microscopy (CLEM) of CORR/Tg^{PB-E3xK-sfGFP} neurons at day 28 was performed using an antibody against total α-syn (LB509). CLEM reveals accumulation of vesicles, lipid droplets, and golgi stacks (*) within regions of the cell that stain positive for α-syn. 7D: Electron microscopy (EM) with GFP-immunogold labeling of day 25 CORR/Tg^{PB-E3xK-sfGFP} neurons confirms the presence of α-syn-sfGFP-positive labeling within inclusions that are rich in lipid droplets.

FIGS. 8A-J. U2OS cellular models. Non-neuronal (U2OS) cellular models generated through our PiggyBac system capture distinct α-syn inclusions. 8A, α-syn structural elements important in our study. 8B-F, U2OS cells expressing (doxycycline-inducible) α-syn A53T or A53T-ΔNAC C-terminally fused to sfGFP, mKATE2 (RFP), exposed to WT α-syn synthetic preformed fibril (PFF; 1-10 μg/mL) and doxycycline (100 ng/ml). 8C, Exposure to PFFs for six days, WB for GFP, mKate2, and α-syn (C20 antibody). In the presence of PFFs, U2OS cells expressing A53T (but not A53T: ΔNAC) reveal α-syn pS129 immunoreactivity (Abcam, ab51253). 8D, Exposure to synthetic PFFs triggers the formation of phosphorylated α-syn (pS129) aggregates that are Triton-insoluble, and can only be extracted by SDS treatment. 8E-F, Number of inclusions in A53T: ΔNAC and A53T cell lines exposed to PFFs (0, 1, 5 and 10 μg/mL). Imaging was performed every two hours for 6 days (IncuCyte custom-made algorithm). Exposure to synthetic PFF seeds α-syn inclusions in a (E) time-dependent and (F, left) dose-dependent manner. Exposure to synthetic PFF decreases the cell density in a dose-dependent manner (F, right). Cells were counted after a 6-day exposure to synthetic PFF, fixed, Hoechst stained, imaged (InCELL2200) and total counts were acquired through ImageJ. Not shown: A53T-PFFs are more potent than WT in this model. 8G, Analogous to A, but expression of sfGFP alone or sfGFP-fused α-syn WT and E3xK. Fluorescent images (sfGFP in green, Hoechst=nuclei in blue) after induction for 96 h (N=2 expts. on different days, 6 wells each: n=12). 8H, WB (α-syn, GFP and actin antibodies) 96 h after dox induction (N=6). 8I, Growth curves (confluence monitored via IncuCyte) for cell lines characterized in G and H: E3xK, but not sfGFP or α-syn WT induction cause marked toxicity (N=2, N=12). 8J, E3xK vs. E3xKΔNAC puncta count in green channel (IncuCyte, custom-made algorithm), plus representative images (210 hours post-dox); N=2, n=12.

FIGS. 9A-B. Example of unbiased screen design in a U2OS model. 9A. In this paradigm, we show a pooled screen format, and the assay is survival. Since α-syn overexpression is not very toxic in this model, the ideal readout will be synthetic lethality (in which the genetic manipulation does not affect control sfGFP cells but is lethal in combination with α-syn overexpression or mutation). In this figure we show pooled CRISPR/Cas9-based screens that cover most genes of the genome with 5 guide RNAs (gRNA) per gene. Cells were infected with the gRNA/Cas9 lentivirus library at low MOI (0.2) with a representation of 500 in triplicate, followed by puromycin (2 μg/mL) selection for 1 week, or until an uninfected control plate completely died. An initial cell pellet was harvested as day 0 after expansion of the puromycin-selected cells to the appropriate scale to begin the screen. Cells were re-plated and treated with doxycycline (100 ng/mL) to induce α-syn. Cell pellets were harvested 7 days and 14 days after doxycycline induction. Genomic DNA was isolated by phenol: chloroform extraction and gRNAs were PCR amplified with barcoded primers for sequencing on an Illumina NextSeq 500. Sequencing reads were aligned to the initial library and counts were obtained for each gRNA. We have completed two CRISPR screens (wild-type α-syn versus E3xK α-syn versus sfGFP; A53T-α-syn versus A53T-ΔNAC-α-syn versus sfGFP). 9B. gRNAs targeting essential genes are depleted in each of the three lines used in the U2OS/Tg^{PB-E3xK-sfGFP} screen, providing quality control validation that the CRISPR gRNA library and screen performed effectively. The log₂ fold-change in read counts from day 14/day 0 is shown. Human essential genes, color-coded in purple, are shifted to the left, as expected for depletion of their gRNAs.

FIGS. 10A-E. PiiN hESC/Tg^{AAVS1} Model. Transgenic α-syn over-expressing hESc lines. 10A. α-syn construct under the TetR responsive promoter was targeted to one allele of the AAVS1 “safe harbor” locus of PPP1R12C. The other allele was targeted with the M2rtTA tetracycline transactivator (CAAGS promoter). 10B. hESc showing tightly regulated and doxycycline dose-dependent expression of α-syn. 10C. Induction of α-syn-mKate2 with doxycycline in an hESc line double-targeted with M2rtTA and α-syn-mKate2 transgene, differentiated to DA neuron-enriched cultures. 10D. Targeted α-syn-mKate2 hESc line transfected with a PiggyBac construct. 10E. List of lines (α-syn and tau transgenes) generated in two hESc backgrounds-WIBR-1 male line and WIBR-3.

FIGS. 11A-C. PiggyBac Induced Astrocytes. 11A. Modified pB vector with an NFIB insert regulated by TRE4G inducible promoter allowing transdifferentiation of iPSCs into astrocytes within 7 days. 11B. IF images of H1 hESC-derived pB-induced astrocytes stained for canonical astrocyte markers GFAP, S100β+, Vimentin and AQP4. Nuclei were stained with Hoechst. 11C. IF quantification across 3 technical replicates. Each replicate represents 42 image fields (1 image field >60 cells) acquired from one well. 2 iPSC lines from multiple system atrophy (MSA) were used to overexpress NFIB to generate induced astrocytes.

FIG. 12. Schematic outline of transgenic αS overexpression driven by STMN2 promoter.

FIG. 13. SNCA-GFP transgene integration (with A53T or ΔNAC mutations) at the STMN2 locus did not alter STMN2 expression as measured by qPCR.

### DETAILED DESCRIPTION

While patient iPSc-derived neurons represent an extraordinary tool to understand neurodegenerative disease, one clear deficiency in these models to date is the lack of inclusion (protein aggregation) pathologies that are the defining hallmark of these diseases. Alpha-synucleinopathies in which α-syn inclusions are found in diverse CNS cell types including cortical glutamatergic and dopaminergic (DA) neurons include Parkinson's disease (PD), Parkinson's disease dementia (PDD), and dementia with Lewy bodies (DLB). In another alpha-synucleinopathy, multiple system atrophy (MSA), there are widespread oligodendroglial and neuronal inclusions (10). In addition, α-syn inclusions are found in >50% of patients with sporadic and familial AD, correlating with cognitive impairment and colocalizing with tau pathology (11). Inclusions all stain avidly for phosphorylated α-syn at serine 129 (α-syn pS129), but they are ultrastructurally diverse. For example, Lewy bodies (LBs) comprise a fibrillar core and are surrounded by vesicles and mitochondria. Pale bodies (PBs) on the other hand comprise a medley of lysosomes, mitochondria and membranous vesicles. Because LBs are sometimes noted to be “extruded” from the periphery of PBs, PBs may be precursors to LBs. A recent publication showed Lewy pathology with correlative light and electron microscopy/tomograph and label-free spectroscopic method. The study confirmed a substantial membrane-rich and high-lipid component to the Lewy pathology (9). Importantly, the correlation of α-syn aggregation pathology in the neurons and glia only loosely correlates with neuronal loss. Some inclusion types may be protective and others detrimental, for example, and different ultrastructural inclusion types may reflect very different biological consequences for the cell.

Even among fibril-rich inclusions, there is considerable diversity. α-Syn can adopt different properties based on differing backbone amino-acid sequence or distinct higher-order amyloid assemblies, a concept originally articulated in the context of prion “strains” and used to explain trans-cellular spread of α-syn from diseased to healthy tissue (5). Distinct α-syn amyloid strains can be generated through differing preparation methods in vitro (buffers, additives, pH, temperature etc.) and these lead to distinct cytopathologies and disease phenotypes in mice (12). There is evidence that synucleinopathies may relate to unique cellular/circuit tropism of distinct α-syn strains. For example, patients with point mutations or gene multiplication in SNCA (encoding α-syn) or GBA (glucocerebrosidase) exhibit diffuse DLB/PDD pathology in the brainstem and cortex. Some α-syn strains have different propensity to cross-fibrillize with the tau protein implicated in AD (13), raising the possibility that specific α-syn conformers underlie mixed AD/DLB pathology. α-syn precipitated from lysates of MSA patient brain is far more transmissible in prion-like fashion to mice over-expressing α-syn than PD lysate (14).

How distinct α-syn inclusions and strains form is not well understood but the host cellular microenvironment may be key. Notably, classical LBs are rarely found in the cortex, but can be found in the basal forebrain (10). LBs are more often found in DA neurons of the midbrain, or the locus coeruleus, raising the possibility that inclusion formation may be related to the host cells in which they form. The same may hold true for α-syn strain formation. For example, the particularly toxic and transmissible strain implicated in MSA forms in an oligodendroglial host environment, but not in neurons (15). These findings underscore the need to study inclusion and strain formation in appropriate host cells. The present methods and compositions advance previous iPSc modeling (8, 16) in which early pathologies were identified in distinct patient-specific CNS cell types but in which inclusion pathologies seen in human postmortem brain were not identified.

The present disclosure addresses the pressing need to rapidly and tractably capture distinct conformations of the aggregation-prone proteins responsible for neurodegeneration in human stem cell-derived CNS models, and provides proof-of-principle for α-syn inclusions. These include pale bodies (PBs), Lewy bodies (LBs), Lewy neurites (LNs) and glial cytoplasmic inclusions (GCIs) and are found in distinct populations of neurons and glia. Ultrastructurally, PBs consist of vesicle membrane-rich structures and LBs consist of fibril-rich structures. Both are generally spherical, while LNs are axonal accumulations of a α-syn into amyloid fibrils of distinct morphology (typically rod-shaped) (17). Cognitive decline and motor dysfunction correlate well with α-syn pathology within cortical glutamatergic neurons and midbrain dopaminergic (DA) neurons, respectively (18). Patients with highly penetrant autosomal dominant α-syn mutations develop early-onset motor dysfunction (“parkinsonism”) and dementia, and polymorphisms at the SNCA locus are among the most common variants associated with PD (19). Certain mutations, for example the “A53T” α-syn mutation, lead to increased propensity for fibrillization, and others like the “E46K” α-syn mutation lead to increased membrane binding in a cellular milieu. An amplified version of the latter mutation known as E3xK has proved to be a useful model to accelerate the toxicity of α-syn in cellular (20) and transgenic mouse models (21). Importantly, simply inheriting extra copies of wild-type SNCA is sufficient to cause early-onset aggressive dementia and parkinsonism, suggesting that over-expression is a valuable way to model these diseases in cellular models. The examples provided herein focus on rapidly induced proteinopathy cortical neuronal models, since these are widely relevant across all known proteinopathies.

Described herein are multiple transgenic systems in which a misfolding-prone protein (e.g., α-syn, beta-amyloid, tau, TDP-43) can be over-expressed efficiently in a distinct neuron or glial cell of the central nervous system. The first transgenic method is with a PiggyBac transposon, a construct with significant modifications (e.g., insertion of insulator UCOE sequences, antibiotic selection genes, and reverse TTK selection genes) that can be used to over-express the target protein of interest. These constructs can be introduced into any cell line to create cellular models of disease that are useful, e.g., for high-throughput genetic and compound screening. These constructs have the advantage of having a high cargo capacity and being virus-free and scalable; the preferred PiggyBac constructs also do not suffer from overproduction inhibition (see Woodard et al. (22)). CNS patient-derived cellular models can thus be created by co-expressing transcription factors that lead to direct conversion (“transdifferentiation”) from induced pluripotent stem cells (iPSc) to neurons or glial cells. For example, co-expression of the Ngn2 transcription factor within the PiggyBac construct in addition to the target protein leads to concomitant trans-differentiation of iPSc to neurons as they over-express the toxic protein of interest. While the PiggyBac approach is powerful, these integrate in numerous possible genomic loci. To counteract this, the target protein can also be inserted into specific locations in the genome by CRISPR/Cas9 or TALEN technology (23). Targeted loci can be so-called safe-harbor loci like AAVS1 (24) or loci that lead to expression in CNS cells, for example the STMN2 locus is relatively neuron-specific.

Thus, described herein are in vitro cellular models of proteotoxicity and proteinaceous inclusions in human cells, and methods for making them. These models can be used, e.g., for investigation of the underlying biology of related neurodegenerative diseases as well as for identification of new therapeutic and/or disease exacerbating agents.

### Cells

A number of different cell types can be used in the present methods and compositions; although human cells are exemplified, other mammalian cells can also be used. Examples include human induced pluripotent stem cells (hiPSc), human embryonic stem cells (hESc), and cells from cultured (e.g., immortalized) cell lines, e.g., HEK/HEK293 cells; HT-1080; U2OS cells; long-term-neuroepithelial stem (lt-NES) cells; and PER.C6 cells (25, 26).

In some embodiments, the cells are iPSc made from cells obtained from a human subject, e.g., a subject who has been diagnosed with a neurodegenerative disease as described herein, e.g., a subject who has a disease-associated mutation as described herein in their genome.

In some embodiments, the cells are made using PiggyBac vectors, which is a scalable technology that frees the system from the need to use viruses. Coupled with co-expression of transcription factors for transdifferentiation into different neurons or glial subtypes, the PiggyBac system avoids limitations associated with variable differentiation protocols such as batch to batch and line to line variability.

In some embodiments, the cells are differentiated or transdifferentiated into neuronal types, thereby reproducing certain subtleties of these neuropathologies. Patients from two α-syn families (i.e., harboring A53T or E46K mutations in α-syn) suffer from Parkinsonism and dementia with midbrain dopaminergic (DA) and cortical glutamatergic neuronal pathology. Protocols for both neuronal types can enable cross-comparisons with patient phenotypes (e.g., asking whether severity of cortical phenotypes is more pronounced in patients with severe dementia versus those with predominant Parkinsonism). Using a cortical transdifferentiation protocol also allows for far more rapid generation of neurons; in some embodiments, a one-step neuronal trans-differentiation protocol through forced expression of the transcription factor Ngn2 (27) is used. This is a rapid protocol, generating mature neurons within 10 days at high levels of purity. In some embodiments, the Ngn2 and the target protein are expressed from a PiggyBac vector, i.e. an all-in-one construct enabling transdifferentiation and overexpression simultaneously. In some embodiments, a target protein encoding sequence can be integrated into either the AAVS1 or STMN2 locus using genome engineering, and then expression of Ngn2, e.g., from an inducible promoter, can be used to transdifferentiate iPS or hES cells into neurons. PiggyBac expression vectors to generate other CNS cell types have been constructed, e.g., for astrocytes (NFIB-Sox9 (28) or NFIA (29) and oligodendrocytes (Sox9) (7) that can be used to drive the expression of the toxic neurodegeneration-associated protein in other CNS cell types. See also Arenas et al. (30) (making DA neurons); Zhang et al. (27) (induction of functional neurons from human pluripotent stem cells).

### Engineering Cell Lines Using Modified Transposon Vectors

Vectors for use in the present methods and compositions include modified transposons, e.g., PiggyBac or Sleeping Beauty (31, 32), that can direct insertion of a transgene into the chromosome of a cell. For example, the construct can include: a coding region; a promoter sequence, e.g., a promoter sequence that restricts expression to a selected cell type, a conditional promoter, or a strong general promoter; an enhancer sequence; untranslated regulatory sequences, e.g., a 5′ untranslated region (UTR), a 3′ UTR; a polyadenylation site; and/or an insulator sequence. Such sequences are known in the art, and the skilled artisan would be able to select suitable sequences. See, e.g., Current Protocols in Molecular Biology, Ausubel, F. M. et al (33) Vancura (ed.) (34) and other standard laboratory manuals. The nucleotide sequence can include one or more of a promoter sequence, e.g., a promoter sequence; an enhancer sequence, e.g., 5′ untranslated region (UTR) or a 3′ UTR; a polyadenylation site; an insulator sequence; or another sequence that increases the expression of an endogenous peptide or increases expression, level, or activity of an endogenous polypeptide.

Exemplary vectors that can be used in the present methods and compositions can include one or more of: sequences for inducible expression (e.g., a bicistronic PiggyBac construct harboring a reverse tetracycline transactivator (35, 36)); a sequence encoding a target protein (e.g., α-syn, APP, TDP-43 or tau genes); a sequence encoding transdifferentiation-mediating transcription factor, e.g., Ngn2 for cortical neuron (e.g., NM_024019.4 (mRNA), encoding NP_076924.1 (protein)); at least one insulator (e.g., iA4 insulator, UCOE insulator, cHS4 insulator, and iA2 insulator); and optionally a detectable tag, e.g., a fluorescent tag (e.g., sfGFP). The system can be fully GATEWAY cloning system (Invitrogen)-compatible allowing versatile expression of any aggregation-prone or neurodegeneration-related protein.

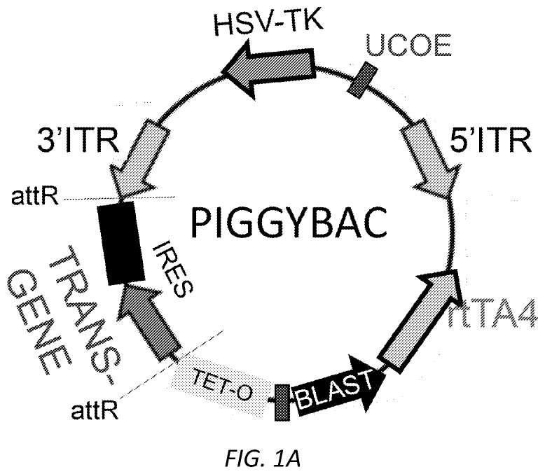

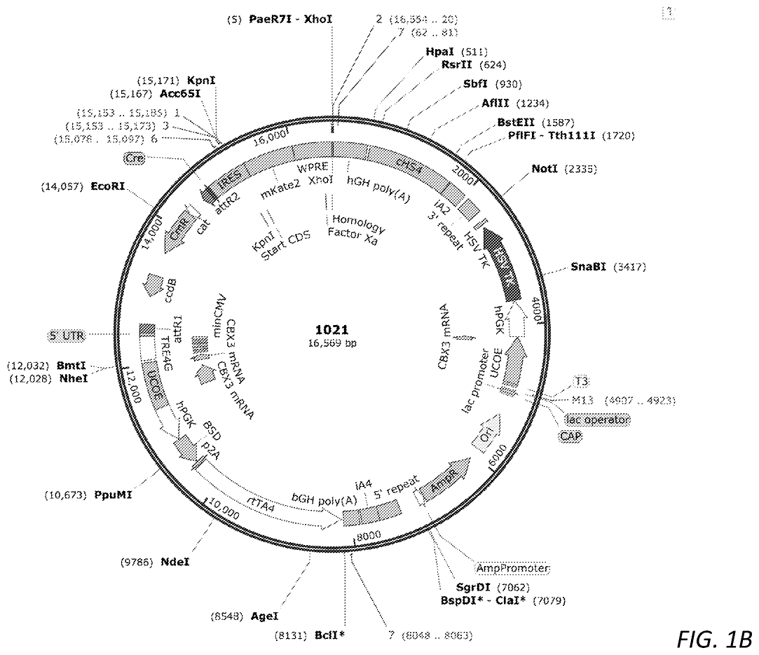

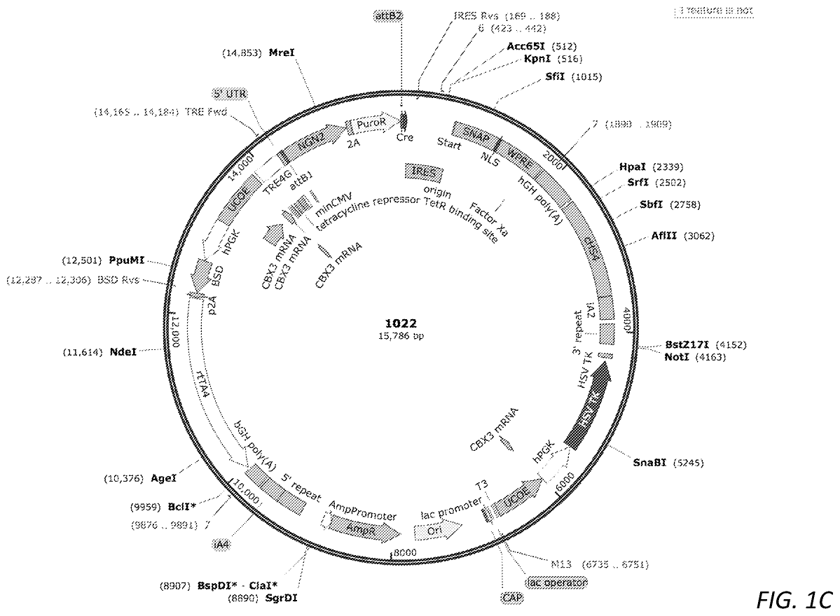

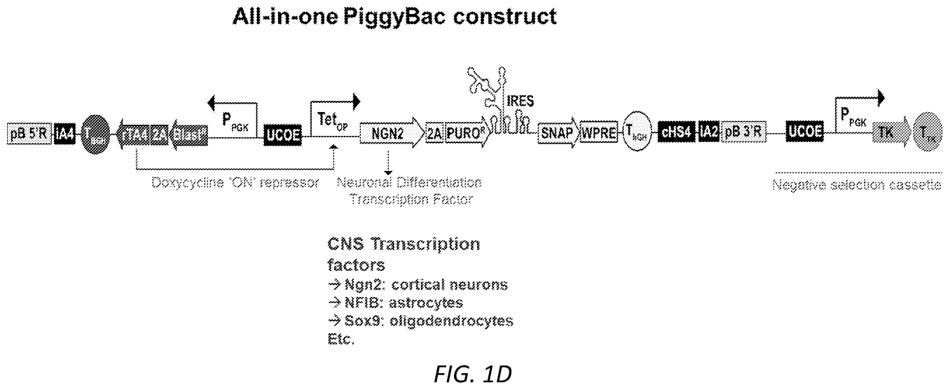

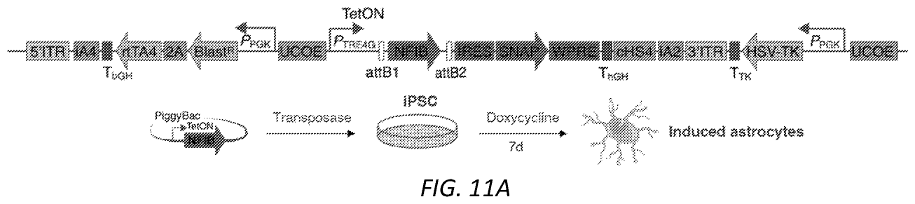

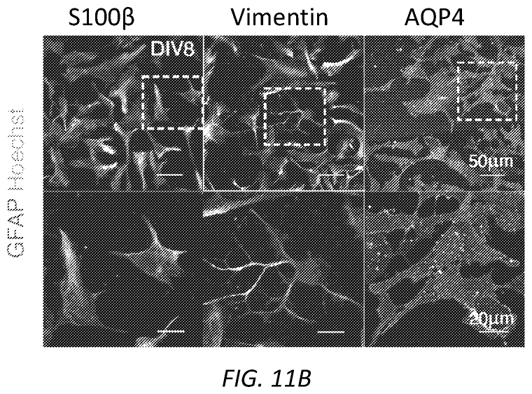

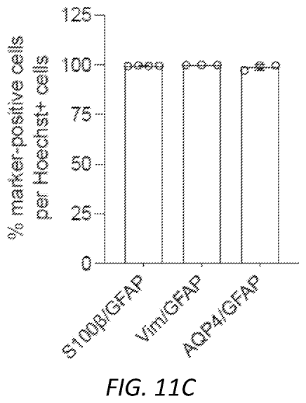

FIGS. 1A-D show exemplary bicistronic modified PiggyBac constructs as described herein. Sequences between the inverted terminal repeats (5′ repeat and 3′ repeat) insert into the genome at PiggyBac insertion “hot spots” containing TTAA target sequence (22). Preferably, these vectors are bicistronic, and have notable features including insulating UCOE sequences, antibiotic selection (e.g., blasticidin in the backbone) and a negative selection cassette (HSV-thymidine kinase/TK) outside of the repeat insertion to guard against spurious insertion. In preferred embodiments, the transgenic insertion site is gateway-cloning compatible (i.e. between attb1 and attb2 sites). The inserted transgene is expressed under a 4th generation rtTA4 reverse tetracycline transactivator system. The vector shown in FIG. 1D includes an Ngn2-2A-Puro insert, allowing for simultaneous Ngn2 expression (to transdifferentiate pluripotent stem cells into cortical neurons), as discussed further below. In place of Ngn2, other transcription factors have been engineered to create different CNS cell types, for example astrocyte (NFIB, FIGS. 11A-C) and oligodendrocyte (Sox9) inducing factors.

### Engineering Cell Lines Using Site-Specific Genome-Editing

Site-specific genome editing through methodologies such as CRISPR/Cas9 gene editing (as well as other methods known in the art, e.g., Zinc Fingers (ZF), Homology directed repair (HDR), or TALEs), can be used to insert sequences coding for target proteins (e.g., α-syn, APP, TDP-43 or tau), optionally sequences comprising disease-associated mutations, or to insert exogenous promoters or disease-associated mutations into endogenous genes, in cells as described herein, e.g., human embryonic cells or pluripotent stem cells of defined genetic background (hESc/hIPSc), or cells from a cultured cell line.

In some embodiments, the target protein encoding sequences can be inserted at specific sites in the genome (e.g., at the AAVS1 or STMN2 loci). STMN2 is a neuron-specific gene, which can allow for relatively neuron-specific expression of the target protein from the STMN2 locus. Integration of the target protein into the AAVS1, a “safe harbor” locus, can be used with inducible expression of the target protein (e.g., by doxycycline).

Exemplary Sequences for Knock-in Loci

| Accession No. | Name |
|---|---|
| AC010327.8 | AAVS1 (adeno-associated virus integration site 1) |
| NM_007029.4 | STMN2 (stathmin 2) |

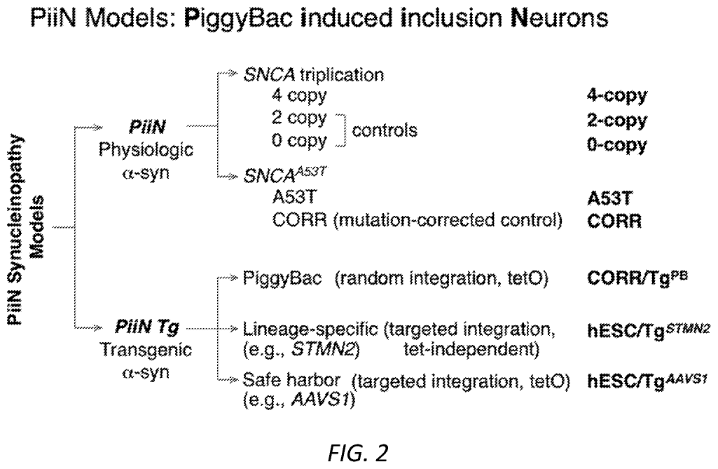

FIG. 2 provides an example of a family of iPSC reagents generated to present proof-of-principle examples herein. The aim is these examples is to model α-synucleinopathies. In these examples, the target cell type is cortical neurons, an important cell type affected in diverse proteinopathies. Thus, a PiggyBac construct encoding Ngn2 (FIGS. 1C-D) is stably introduced into each iPSC line, enabling transdifferentiation into cortical neurons. These reagents are known thus as “PiiN” reagents (for PiggyBac Ngn2 induced inclusion neurons). In some cases, α-syn expression is at physiologic levels, e.g. in the case of the α-syn triplication (4-copy) or mutant A53T lines (together with mutation-corrected controls). These lines are known as 4-copy, 2-copy, 0-copy and A53T/CORR lines, respectively. In other cases (“PiiN Transgenic” models), to greatly enhance the extent of aggregation pathology, we coupled Ngn2 expression with transgenic over-expression of α-syn, either through random integration with an all-in-one bicistronic PiggyBac vector (Tg^{PB}), or through targeted integration at safe-harbor loci, e.g. AAVS1 (Tg^{AAVS1}) or lineage-specific loci, e.g. STMN2 (Tg^{STMN2}).

### Target Proteins

Target proteins that can be used in the present methods and compositions include α-syn, APP, TDP-43 (also known as TARDBP), or tau.

Exemplary Sequences for Human α-syn

| mRNA | Protein | Note |
|---|---|---|
| NM_000345.3 | NP_000336.1 | variant (1, also known as NACP140) is |
| | | the longest transcript and encodes the |
| | | longer isoform (NACP140). Variants 1, |
| | | 2, and 3 all encode the same isoform |
| NM_001146054.1 | NP_001139526.1 | variant (2) differs in the 5′ UTR |
| | | compared to variant 1 |
| NM_001146055.1 | NP_001139527.1 | variant (3) differs in the 5′ UTR |
| | | compared to variant 1. Variants 1, 2, and |
| | | 3 all encode the same isoform |
| | | (NACP140) |
| NM_007308.2 | NP_009292.1 | variant (4, also known as NACP112) |
| | | lacks an alternate in-frame exon |
| | | compared to variant 1. The resulting |
| | | isoform (NACP112) has the same N- |
| | | and C-termini but is shorter compared to |
| | | isoform NACP140 |

Exemplary Sequences for Human tau

| mRNA | Protein | Note |
|---|---|---|
| NM_001123066.3 | NP_001116538.2 | variant (6) encodes the longest isoform |
| | | (6) |
| NM_001123067.3 | NP_001116539.1 | variant (5) lacks four internal coding |
| | | exons, as compared to variant 6. The |
| | | reading frame is not affected, and the |
| | | resulting isoform (5) has identical N- |
| | | and C-termini but lacks four segments, |
| | | as compared to isoform 6 |
| NM_001203251.1 | NP_001190180.1 | variant (7) lacks four internal coding |
| | | exons, as compared to variant 6. The |
| | | reading frame is not affected, and the |
| | | resulting isoform (7) has identical N- |
| | | and C-termini but lacks four segments, |
| | | as compared to isoform 6 |
| NM_001203252.1 | NP_001190181.1 | variant (8) lacks three internal coding |
| | | exons, as compared to variant 6. The |
| | | reading frame is not affected, and the |
| | | resulting isoform (8) has identical N- |
| | | and C-termini but lacks three segments, |
| | | as compared to isoform 6 |
| NM_005910.5 | NP_005901.2 | variant (2) lacks three internal coding |
| | | exons, as compared to variant 6. The |
| | | reading frame is not affected, and the |
| | | resulting isoform (2) has identical N- |
| | | and C-termini but lacks three segments, |
| | | as compared to isoform 6 |
| NM_016834.4 | NP_058518.1 | variant (3) lacks five internal coding |
| | | exons, as compared to variant 6. The |
| | | reading frame is not affected, and the |
| | | resulting isoform (3) has identical N- |
| | | and C-termini but lacks four segments, |
| | | as compared to isoform 6 |
| NM_016835.4 | NP_058519.3 | variant (1) lacks one internal coding |
| | | exons, as compared to variant 6. The |
| | | reading frame is not affected, and the |
| | | resulting isoform (1) has identical N- |
| | | and C-termini but lacks one segment, |
| | | as compared to isoform 6 |
| NM_016841.4 | NP_058525.1 | variant (4) lacks six internal coding |
| | | exons, as compared to variant 6. The |
| | | reading frame is not affected, and the |
| | | resulting isoform (4) has identical N- |
| | | and C-termini but lacks five segments, |
| | | as compared to isoform 6 |

Exemplary Sequences for Human APP

| mRNA | Protein | Note |
|---|---|---|
| NM_000484.4 | NP_000475.1 | variant (1) represents the longest |
| | | transcript and encodes the longest |
| | | isoform (a, also known as PreA4 770) |
| NM_001136016.3 | NP_001129488.1 | variant (4) differs in the 5′ UTR and |
| | | coding sequence and lacks an alternate |
| | | in-frame exon compared to variant 1. |
| | | The resulting isoform (d) has a shorter |
| | | and distinct N-terminus and lacks an |
| | | internal segment compared to isoform a |
| NM_001136129.3 | NP_001129601.1 | variant (5) lacks three alternate in-frame |
| | | exons compared to variant 1. The |
| | | resulting isoform (3) has the same N- |
| | | and C-termini but is shorter compared to |
| | | isoform a |
| NM_001136130.3 | NP_001129602.1 | variant (6) lacks an alternate in-frame |
| | | exon compared to variant 1. The |
| | | resulting isoform (f) has the same N- |
| | | and C-termini but is shorter compared to |
| | | isoform a |
| NM_001136131.2 | NP_001129603.1 | variant (7) differs in the 5′ UTR and |
| | | coding sequence and lacks two alternate |
| | | in-frame exons compared to variant 1. |
| | | The resulting isoform (g) is shorter at |
| | | the N-terminus and lacks an internal |
| | | segment compared to isoform a |
| NM_001204301.2 | NP_001191230.1 | variant (8) lacks an alternate in-frame |
| | | exon compared to variant 1. The |
| | | resulting isoform (h, also known as L- |
| | | APP752) has the same N- and C-termini |
| | | but is shorter compared to isoform a. No |
| | | full-length transcript is available for this |
| | | transcript; however, it is supported by |
| | | PMID: 1429732 and other publications |
| NM_001204302.2 | NP_001191231.1 | variant (9) lacks two alternate in-frame |
| | | exons compared to variant 1. The |
| | | resulting isoform (i, also known as L- |
| | | APP733) has the same N- and C-termini |
| | | but is shorter compared to isoform a. No |
| | | full-length transcript is available for this |
| | | transcript; however, it is supported by |
| | | PMID: 1429732 and other publications |
| NM_001204303.2 | NP_001191232.1 | variant (10) lacks three alternate in- |
| | | frame exons compared to variant 1. The |
| | | resulting isoform (j, also known as L- |
| | | APP677) has the same N- and C-termini |
| | | but is shorter compared to isoform a. No |
| | | full-length transcript is available for this |
| | | transcript; however, it is supported by |
| | | PMID: 1429732 and other publications |
| NM_201413.3 | NP_958816.1 | variant (2) lacks an alternate in-frame |
| | | exon compared to variant 1. The |
| | | resulting isoform (b, also known as |
| | | PreA4 751) has the same N- and C- |
| | | termini but is shorter compared to |
| | | isoform a |
| NM_201414.3 | NP_958817.1 | variant (3) lacks an alternate in-frame |
| | | segment compared to variant 1. The |
| | | resulting isoform (c, also known as |
| | | PreA4 695) has the same N- and C- |
| | | termini but is shorter compared to |
| | | isoform a |

Exemplary Sequences for Human TARDBP/TDP-43

| mRNA | Protein | Note |
|---|---|---|
| NM_007375.4 | NP_031401.1 | — |

### Disease-Associated Mutations

The present methods can include using cells that have, or are engineered to have, disease-associated mutations in a target protein. For example, a disease-associated mutation can include a mutation in the lipophilic protein α-syn, which is tied to Parkinson's disease (PD) and dementia with Lewy bodies (DLB). Increased gene dosage of wild-type α-syn, or point mutations at the α-syn locus (e.g., A30P, A53T, E46K, G51D) lead to aggressive dominantly inherited forms of PD/DLB. “E3xK” refers to the ‘3xE46K amplification’ model (E35K+E46K+E61K) that “amplifies” the effect of the E46K mutation, resulting in enhanced membrane affinity and a toxic form of α-syn associated with stalled vesicle (but amyloid-free) inclusions. As noted above, the hallmark pathology of PD and DLB comprises α-syn-predominant inclusions, known as Lewy bodies (LBs), within degenerating cells, including dopaminergic (DA) and cortical neurons. LBs are intracytoplasmic inclusions rich in α-syn amyloid fibers, but also surrounded by clustered vesicles. α-syn/vesicle clusters are considered LB precursor structures. These ultrastructural features parallel strong interest in both amyloid and vesicle-trafficking pathologies in the PD field. In MSA, another synucleinopathy, both nuclear and cytoplasmic α-syn-predominant inclusions occur in widespread neuronal populations but the hallmark pathology is α-syn-predominant glial cytoplasmic inclusions (GCIs) in oligodendrocytes. There is no known mutation in α-syn that predisposes to MSA although G51D and α-syn triplication patients do exhibit prominent GCIs in parts of the brain.

Other disease-associated mutations include mutations in APP, tau, or related proteins that lead to the formation of proteotoxic or proteinaceous inclusions in the cell. In some embodiments, mutations in the tau protein can include G272V, N279K, P301L, ΔK280, and V337M (von Bergen, et al., JBC, Oct. 17, 2001; 276, 48165-48174); mutations R5L, K257T, 1260V, L266V, G272V, N279K, ΔK280, L284L, ΔN296 N296H, P301L, P301S, S305N/S, L315R, S320F, Q336R, V337M, E342V, S352L, K369I, G389R, and R406W in the tau protein are associated with disease including frontotemporal dementia and parkinsonism linked to chromosome 17 (FTDP-17).

Mutations in the APP gene that may be associated with CAA, AD, PD, PDD, or other neurodegenerative diseases include A201V; A235V; D243N; E246K; E296K; P299L; R468H; A479S; K496Q; A500T; Y538H; V562I; E599K; T600M; P620A; P620L; T663M; E665D; KM670/671NL; (Swedish); A673T; (Icelandic); A673V; H677R; (English); D678H; (Taiwanese); D678N; (Tottori); E682K; (Leuven); K687N; A692G; (Flemish); E693del; (Osaka, E693A, E693delta); E693G; (Arctic, E22G); E693K; (Italian); E693Q; (Dutch); D694N; (Iowa); L705V; G708G; G709S; A713T; A713V; T714A; (Iranian); T714I; (Austrian); V715A; (German); V715M; (French); I716F; (Iberian); I716M; I716T; I716V; (Florida); V717F; (Indiana); V717G; V717I; (London); V717L; T719N; T719P; M722K; L723P; (Australian); K724N; (Belgian); H733P; IVS17 83-88delAAGTAT; c.*18 C>T; c.*331_*332del; c.*372 A>G (numbering with regard to NP_000475.1) (37-41).

Mutations in the TARDBP gene have been found to cause amyotrophic lateral sclerosis (ALS), as well as frontotemporal dementia (FTD) without features of amyotrophic lateral sclerosis (ALS). Pathogenic mutations can include MET337VAL; GLN331LYS; GLY294ALA; GLY290ALA; GLY298SER; ASP169GLY; GLY348CYS; GLN343ARG; ALA315THR; GLY295SER; LYS263GLU; 2076G-A, 3-PRIME UTR; and ALA382THR (42-44).

As noted above the cells used in the present methods can either be cells that already have a disease-associated mutation in the genome (e.g., a cell from a subject who has the mutation and optionally has been diagnosed with the disease or risk of developing the disease that is above the level of risk of the general population), or can be engineered to have the mutation using methods known in the art (e.g., using known recombinant methods including CRISPR, TALEN, or ZF-directed genome engineering, or by stable integration of a sequence comprising the disease-associated mutation into the genome of the cell).

### Distinct Conformational Protein “Strains” and Inclusions Contribute to Heterogeneity of Neurodegenerative Diseases

The host-variant and host-strain phenomena may extend beyond prion diseases to more common degenerative proteinopathies; see, e.g., Jarosz and Khurana (5). For example, aggregation of the wild-type tau protein leads to Pick's disease, progressive supranuclear palsy, and corticobasal degeneration. Each disease exhibits distinct ultrastructural features of tau fibers, cellular and circuit pathologies, and clinical presentations. Similarly, synucleinopathies—including Parkinson's disease (PD), dementia with Lewy bodies (DLB), and multiple system atrophy (MSA)—result from misfolding and mislocalization of the same protein, alpha-synuclein (α-syn), with predilection for distinct cell types and circuits in the nervous system. Point mutations or multiplication at the α-syn-encoding SNCA locus lead to highly penetrant forms of neurodegenerative diseases, with some mutations predisposing to motor symptoms (parkinsonism) followed by later-onset dementia and others to earlier dementia.

Transgenic amyloid-precursor-protein (APP)-overexpressing mice can be induced to seed beta-amyloid in distinct patterns when injected with Aβ-containing brain extracts derived from different hosts (45). Likewise, different conformers of tau and α-syn prepared from synthetic monomer lead to highly distinct yet stereotyped patterns of neurodegeneration when seeded directly into mouse brain (12, 46). Postmortem brain material from MSA patients has been shown to be more effective at seeding α-syn in transgenic mice and cell lines than material from PD or DLB patients (14), and specific cellular environments may be critical for engendering distinct strains (15). These findings have collectively raised the possibility that, just as with PrP, distinct conformer strains of tau and α-syn exist and that the distinct clinical patterns of neurodegenerative proteinopathies may relate to tropism of these strains for distinct cells and circuits within the brain.

Beyond distinct conformations of proteins, proteinaceous inclusions that form in the brain also exhibit diverse ultrastructural characteristics that may be biologically distinct. Alpha-synuclein offer a case in point. Lewy bodies (LBs) comprise amyloid beta-sheet rich fibrils surrounded by a halo rich in vesicle membranes (47). Pathologic α-syn accumulation in LBs and Lewy neurites (LNs) is defined by ubiquitination, phosphorylation of α-syn at S129, proteinase K resistance and staining with amyloid dyes (e.g. thioflavin S) (48). Other α-syn inclusions, that may be precursor lesions to LBs, are known as pale bodies (PBs). These are looser structures with α-syn-containing filaments and abundant vesicle membranes (49). Neuronal and glial inclusions in MSA also consist of filamentous structures, ultrastructurally distinct from each other and also from LBs and PBs found in PD and DLB.

Cells in culture generated using methods described herein can be induced to form inclusions reminiscent of these found in postmortem brain. To trigger filamentous inclusions, pre-formed α-syn fibrils can be introduced into cultured cells whereupon the inclusions, pre-formed fibrils PFFs) can be introduced into cells. These self-template on to α-syn that is either endogenously expressed in the host cell or over-expressed in a transgene. To trigger inclusions that exhibit conformers mimicking those aggregating in patient brains, brain lysates or α-syn PFFs amplified from this material (through a process known as cyclic amplification) can be introduced into cells. In principle, this can result in a powerful model system in which the appropriate host cell and genotype is brought together with the patient-matched α-syn conformer. Exactly analogous methods can be applied to other aggregation-prone proteins.

Specifically, when cells in culture generated using a method described herein were seeded with synthetic or brain-derived α-syn PFFs (50), intracellular inclusions formed that exhibited key features of aggregated α-syn in postmortem human brain, including triton-insolubility, ubiquitination, phosphorylation at Ser129, and dependence on host-cell expression of the “non-amyloid B-component” (NAC) domain of α-syn that is associated with oligomerization and aggregation of α-syn. Moreover, the inclusions that form are ultrastructurally highly reminiscent of inclusions found in human patient brain indicating that these cells are a clinically relevant model of human synucleinopathy including PD.

### Methods of Use

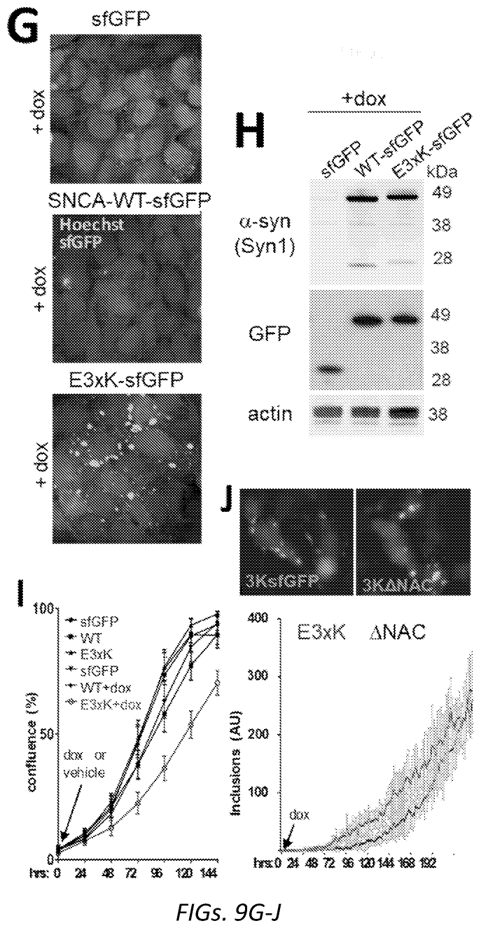

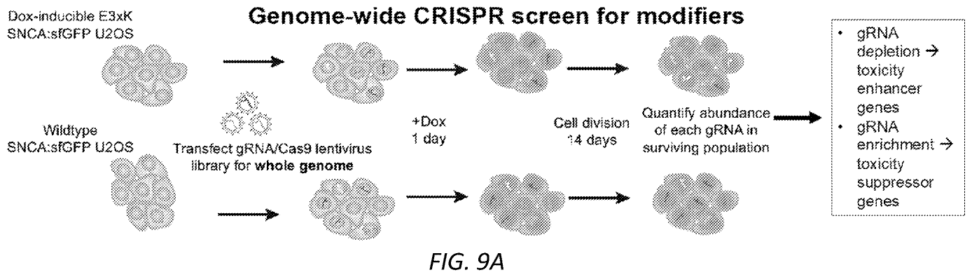

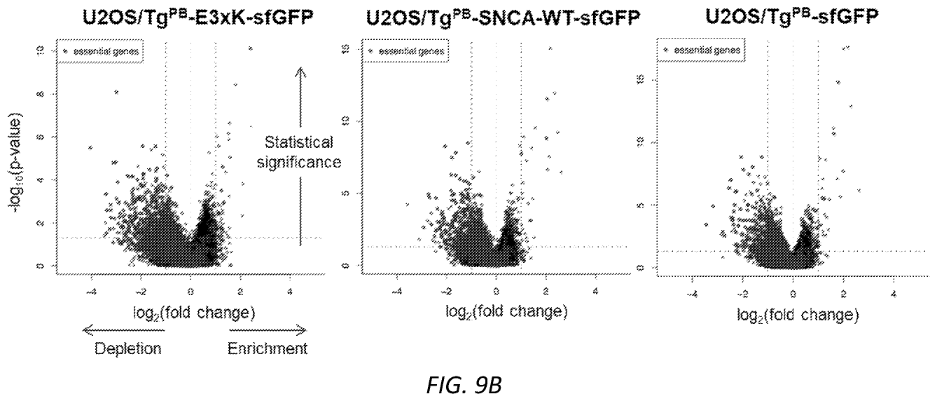

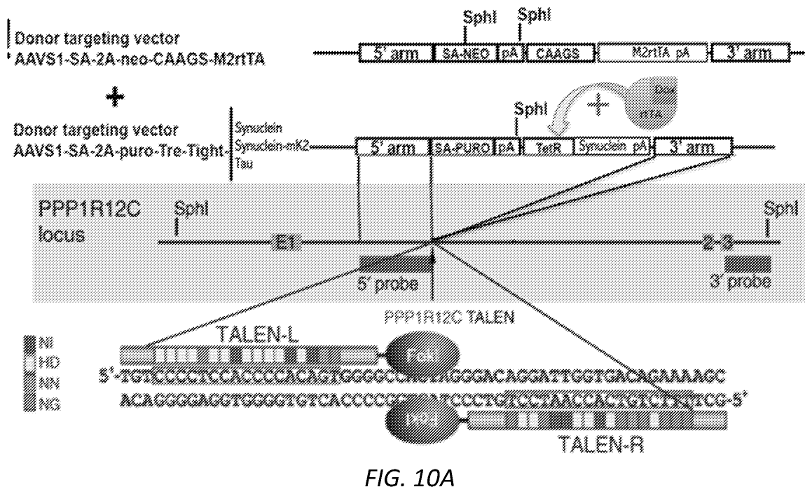

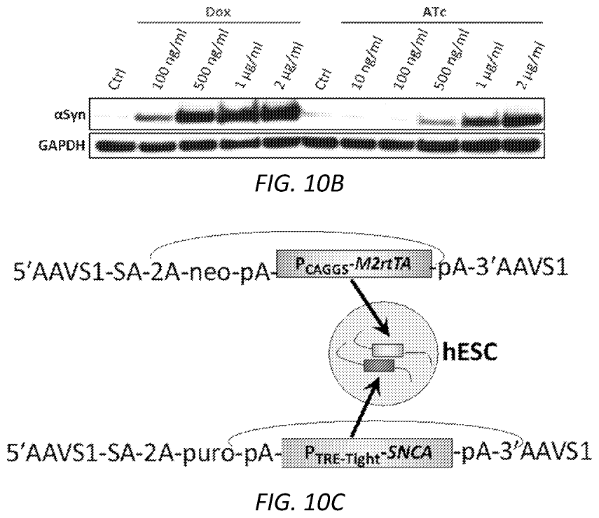

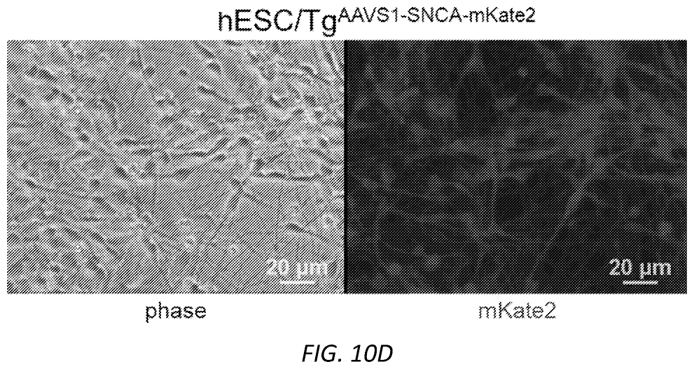

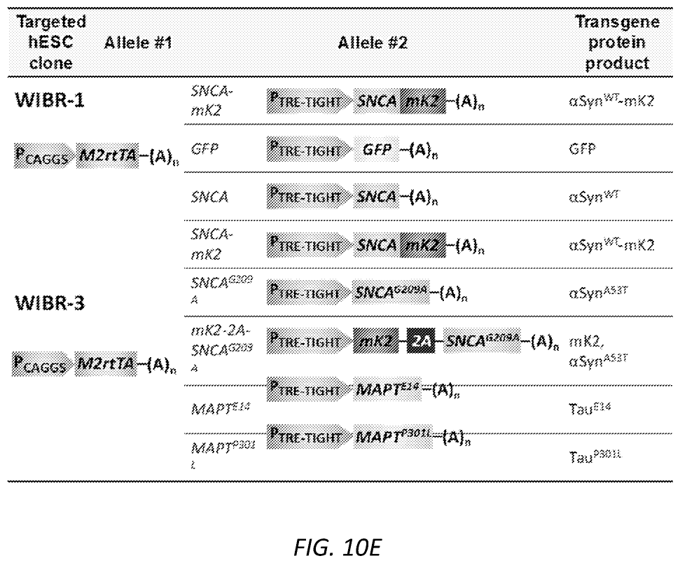

Included herein are methods that will facilitate screening test compounds, e.g., polypeptides, polynucleotides, inorganic or organic large or small molecule test compounds, and antibodies to identify agents useful in the treatment of disorders associated with proteotoxicity and proteinaceous inclusions, e.g., AD, PD, MSA, DLB, ALS and FTDP-17). One example of a screen is shown in FIG. 9, which illustrates an unbiased screen design in a model described herein, e.g., the U2OS model. In this paradigm, we show a pooled screen format, and the assay is survival. Since α-syn overexpression is not very toxic in this model, the ideal readout is synthetic lethality (in which the genetic manipulation does not affect control sfGFP cells but is lethal in combination with α-syn-sfGFP). FIG. 9 shows pooled CRISPR/Cas9-based screens that cover most genes of the genome with 5 guide RNAs per gene. We have completed initial screens for α-syn, in this case within an amplified E→K mutation (E3xK) compared to an sfGFP control. We have completed two CRISPR screens now (wild-type α-syn versus E3xK α-syn versus sfGFP; A53T α-syn versus A53T-ΔNAC α-syn versus sfGFP).

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo (51), and include those such as the “split and pool” or “parallel” synthesis techniques, solid-phase and solution-phase techniques, and encoding techniques (52). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample, e.g., a cell as described herein, optionally in the presence of exogenous fibrils such as the PFFs or sPFFs described herein, and one or more effects of the test compound is evaluated. In a cultured or primary cell for example, the ability of the test compound to reduce proteotoxicity and proteinaceous inclusions in the presence of the fibrils is evaluated, or the effect of the test compound on viability in the presence of the fibrils. FIG. 9 provides an example of unbiased screen design in a U2OS model. In this paradigm, a pooled screen format is used, and the assay is survival.

In some embodiments, a test compound is a therapeutic oligonucleotide, e.g., an antisense RNA, antisense DNA, chimeric antisense oligonucleotides, antisense oligonucleotides comprising modified linkages, interference RNA (RNAi), short interfering RNA (siRNA); a microRNA (miRNA); a small, temporal RNA (stRNA); or a short, hairpin RNA (shRNA); small RNA-induced gene activation (RNAa); small activating RNAs (saRNAs), or combinations thereof (53, 54).

In some embodiments, a test compound is an antibody, e.g., directed at an aggregation-prone protein found in the inclusions (55, 56). Alternatively, the test compound can be an immunotherapy, e.g., as described in Valera et al. (57).

In some embodiments, a test compound is a candidate small-molecule that binds to aggregated forms of misfolding proteins with a view to clinically developing that molecule into a diagnostic radiotracer. Described herein are cellular models that capture both patient-specific cells and patient-specific conformations of aggregation-prone proteins. These thus offer ideal screenable platforms to stratify candidate radiotracers and test them for disease and patient specificity.

Methods for evaluating each of these effects are known in the art. For example, ability to modulate expression of a protein can be evaluated at the gene or protein level, e.g., using quantitative PCR or immunoassay methods. In some embodiments, high throughput methods, e.g., protein or gene chips as are known in the art (58-62), can be used to detect an effect on proteotoxicity and proteinaceous inclusions. Ability to modulate signaling via the cellular trafficking pathway can be evaluated, e.g., using biochemical assays (8), and/or using oxidative stress assays (63).

A test compound that has been screened by a method described herein and determined to reduce proteotoxicity and proteinaceous inclusions, can be considered a candidate compound. A candidate compound that has been screened, e.g., in an in vivo model of a disorder, e.g., Parkinson's disease (PD) and dementia with Lewy bodies (DLB) and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that reduce proteotoxicity and proteinaceous inclusions) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating disorders associated with proteotoxicity and proteinaceous inclusions, as described herein, e.g., Parkinson's disease (PD) and dementia with Lewy bodies (DLB). A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of a neurodegenerative disease, e.g., Parkinson's disease or dementia with Lewy bodies, AD, or FTD, as known in the art or described herein. The animal can be monitored for a change in the disorder, e.g., for an improvement in a parameter of the disorder, e.g., a parameter related to clinical outcome. In some embodiments, the parameter is reduction or prevention of proteotoxicity and proteinaceous inclusions, and an improvement would be cell survival. In some embodiments, the subject is a human, e.g., a human with a neurodegenerative disease, e.g., Parkinson's disease or dementia with Lewy bodies, AD, or FTD, and the parameter is cognitive and/or motor function.

### EXAMPLES

Generation of a Modified all-In-One PiggyBac Plasmid

Into the 1018 plasmid (Addgene) the following was inserted between the piggybac inverted repeats in 5′->3′ order: 5′ PiggyBac ITR, iA4 insulator sequence, bGH poly(A) signal, Blasticidin cassette-2A linker-rtTA4 CDS (2A: self-cleaving peptide), hPGK promotor, UCOE insulator, Tet Response Promotor, Gateway Cloning Cassette, Internal Ribosome Entry Site, NGN2 CDS-2A linker-Puromycin cassette, Woodchuck Proximal Response Element, hGH polyA signal, cHS4 insulator, iA2 insulator, 3′ PiggyBac ITR. A Thymidine Kinase expression cassette was inserted into the backbone of the plasmid for negative selection of the non-transposon random insertions.

### Example 1. Modified PiggyBac Expression Vectors

The 1018 plasmid from Addgene was used as a starting point, and plasmids 1021 and 1022 were generated by first inserting the following between the PiggyBac inverted repeats in 5′->3′ order:
5′ PiggyBac ITR
iA4 insulator sequence
bGH poly(A)
Blasicidin cassette-2A linker-rtTA4 CDS
hPGK promotor
UCOE insulator (see Müller-Kuller et al., Nucleic Acids Res. 2015 Feb. 18; 43 (3): 1577-1592)
Tet Response Promotor
Gateway Cloning Cassette
Internal Ribosome Entry Site
NGN2 CDS-2A linker-Puromycin cassette
Woodchuck Proximal Response Element
hGH polyA
cHS4 inulator
iA2 insulator
3′ PiggyBac ITR

In addition, a Thymidine Kinase expression cassette was inserted into the backbone of the plasmid. Shown in FIG. 1B is Plasmid #1021, an exemplary non-neuronal expression construct for high-throughput screening purposes, which can be used used to generate mutant α-syn-A53T (or the control α-syn A53T missing the pro-aggregation NAC domain “A53T-ΔNAC”). An mKate2 sequence in this example was used after the IRES sequence to label the cells that have integrated the vector with a red fluorescent protein.

Shown in FIG. 1C is exemplary Plasmid #1022, a neuronal construct with Ngn2-2A-Puro insert, allowing for simultaneous Ngn2 expression (to transdifferentiate pluripotent stem cells into cortical neurons) and second antibiotic (puromycin) selection. A SNAP-TAG (New England Biolabs), a protein tag that forms a highly stable, covalent thioether bond with fluorophores or other substituted groups when appended to benzylguanine, was used in this exemplary plasmid after the IRES sequence to label the cells that have integrated the vector. This enables a large selection of substrates for fluorescent labeling of cells for immunofluorescence or FACS sorting. FIG. 1D provides a linearized schematic of the plasmid #1022 depicted in FIG. 1C). Note that, as indicated in FIG. 1C, other cell types can be generated by over-expression of other transcription factors. For example, constructs for transdifferentiation to astrocytes (NFIB) and oligodendrocytes (SOX9) have been generated with the same backbone.

### Example 2: PiggyBac Expression System for Rapid One-Step Generation of Cortical Neurons Expressing α-Syn Mutations or Gene Duplication with Isogenic Controls

A system was developed to enable rapid crossover from human cell-line models directly to superficial layer cortical glutamatergic neurons, a class of neuron affected in synucleinopathy (18). PiggyBac vector enabled tet-inducible expression of Ngn2 transgene, resulting one-step transdifferentiation into superficial glutamatergic neurons within 7 to 8 days through co-expression of Ngn2, a method previously established with viral transduction (27). This is shown in schematically in FIG. 2 (PiiN models). We optimized the protocol to efficiently produce neurons from multiple hESc and hIPSc lines (including iPSC from carriers of mutations in α-syn, shown in FIG. 2; see Example 6). Similar methods were used to generate astrocytes; pB-NFIB (pB-NFIB) expression in hESC resulted in appropriate cell type-specific markers-GFAP, S100ß, vimentin, and AQP4 (FIGS. 11A-B, see also Example 6).

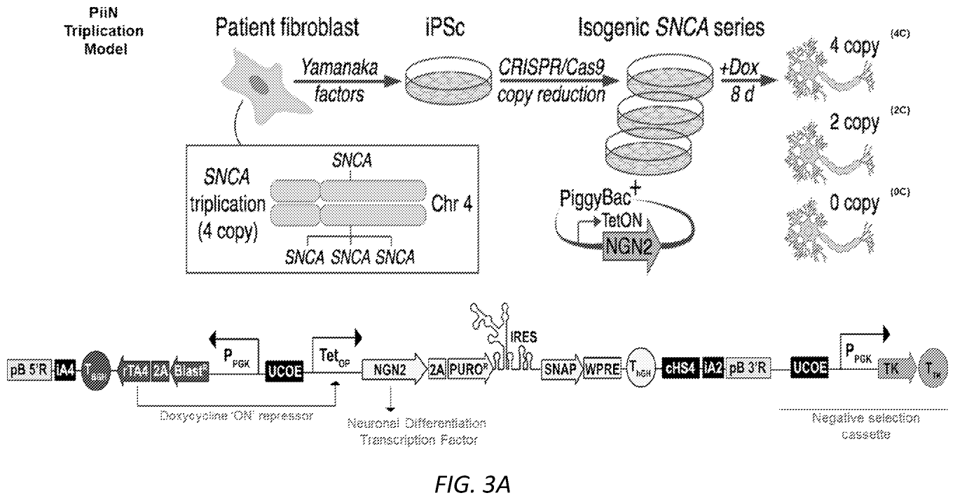

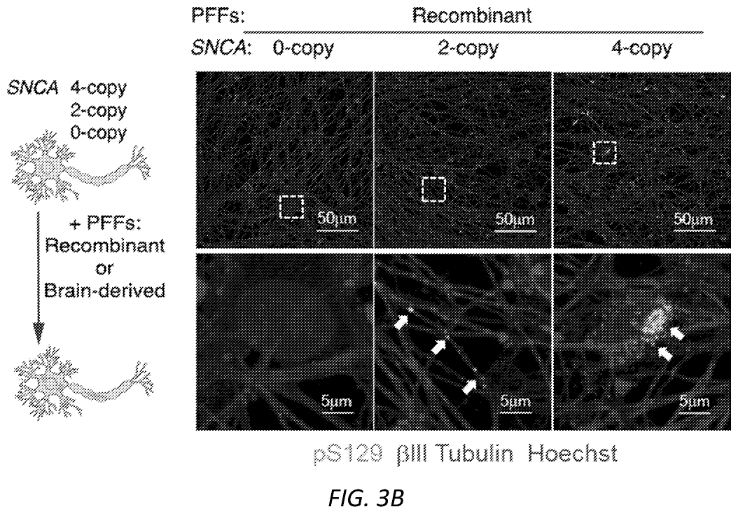

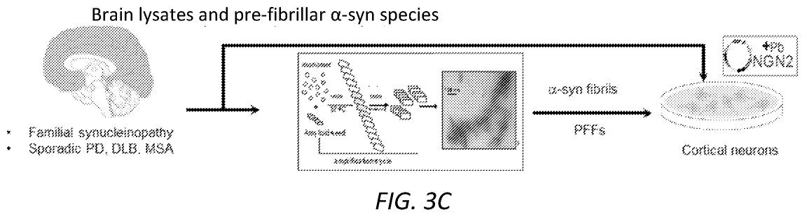

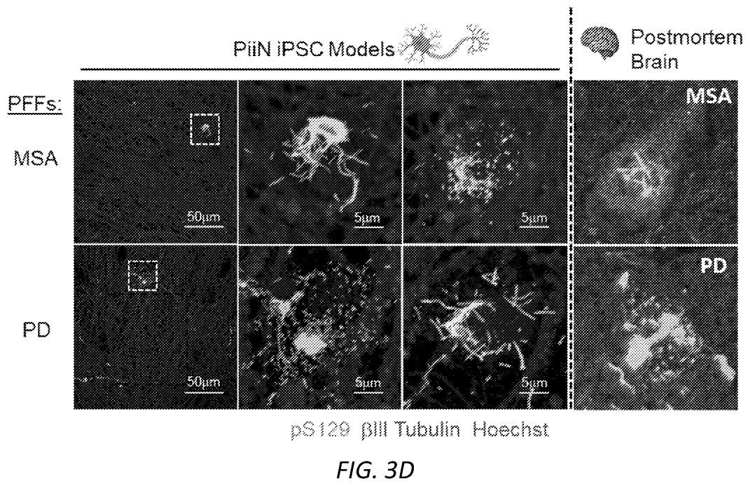

In FIG. 3A, we depict the generation of PiiN reagents from iPSC harboring differential SNCA copy numbers. Briefly, fibroblasts from a PD patient (Iowa kindred) harboring triplication of the SNCA locus were reprogrammed to iPScs by introduction of Yamanaka factors through mRNA-based reprogramming. At the iPSc stage, SNCA copy number was lowered by CRISPR/Cas9 editing to 2-copies (reduced copy, i.e. WT levels) and 0-copies (i.e. complete knockout).

After generation of isogenic lines of copy number series, Ngn2 gene along with the TET-ON system was introduced to each series by an all-in-one PiggyBac vector mediated transposition. The cells were trans-differentiated to cortical neurons by doxycycline induced expression of Ngn2 (induced neurons; iNs). The cells were aged for three weeks (21 days in vitro; 21 DIV) or more (FIG. 3A).

We have obtained approximately 100 pluripotent stem cell lines, from hESCs to hiPSCs, across a number of different neurodegenerative diseases including familial PD, sporadic LBD, and MSA. To date we have successfully introduced pB-NGN2 into 34 iPSC lines and derivative clones and pB-NFIB into 3 iPSC lines.

More specifically, the methods used for genetic correction of α-syn triplication in iPSC lines were as follows.

### CRISPR Design

Isogenic SNCA knock out controls were obtained using the CRISPRs/Cas9 system. Guide RNAs targeting exon 2 of the SNCA gene were designed at crispr.mit.edu/. The gRNAs were cloned into PX458 (Addgene, plasmid #62988), a single plasmid containing both sgRNA and the Cas9 (pSpCas9 (BB)-2A-GFP, following the protocol (Ran, et al, 2013, Nature Protocols). The CRISPR methods were then tested in 293T cells and cutting efficiency was determined by Sanger sequencing and TIDE analysis (tide.deskgen.com).

### Transfection

iPS cells were cultured to 70% confluency and dissociated into single cells using Accutase (StemCell Technologies 07920). Washed cells with DMEM/F12 1:1 medium to remove Accutase. 1.0×10⁶ cells were transfected with 2.5 μg of the CRISPR/Cas9 plasmid PX459 using Lipofectamine 3000 Transfection Reagent (ThermoFisher L3000015). Briefly, prepared DNA-lipid complex followed the Lipofectamine 3000 Reagent Protocol and incubated at room temperature for 15 minutes. Resuspended the single cells to a minimal volume (e.g., 1.0×10⁶ cells in about 50 μl medium) and drop-wisely added the DNA-lipid complex to the cells. Mixed by gently flicking the tube wall 2-3 times. Incubated at room temperature for another 10 minutes. Added 2 ml prewarmed mTeSR1 medium supplemented with 10 μM Rock inhibitors and plated the cells to one well of a Matrigel-coated 6-well plate. 48-hour post transfection, cells were subjected to cell sorting and GFP positive cells were collected and plated at clonal density (5 k to 10 k cells per 10 cm dish). In about 7-10 days, colonies were picked into 96-well plate and expanded for genotyping. In total, 60 clones were selected for further analysis.

### Genotyping

DNA for genotyping was extracted using the prepGEM® DNA Extraction Kits (ZyGem PT10050). PCR genotyping was performed using Phusion Green Hot Start II High-Fidelity DNA Polymerase (ThermoFisher F537) following the manufacturer's instructions at an annealing temperature of 62° C. The following screening primers were designed flanking the CRISPR targeted SNCA exon2 site: fwd 5′TAGCCAAGATGGATGGGAGATG (SEQ ID NO:1) and rvs 5′CCATCACTCATGAACAAGCACC (SEQ ID NO:2), which was also used for Sanger sequencing. The indel rate was >80%, 19 clones with indels resulting in significant deletions and/or potential ORF shifts were further investigated via TOPO cloning using TOPO TA Cloning Kit for Subcloning (ThermoFisher 450641), single clone PCR and Sanger sequencing.

### Evaluation of Knock Out Level

Candidate knock out clones were transfected with PiggyBac TRE-NGN2-puromycin, then transdifferentiated to neurons (as described below). SNCA expression level were determined by qRT-PCR using TaqMan primer Hs00240906_m1 and Western blotting using a monoclonal Antibody to α-syn (4B12) (ThermoFisher MA1-90346).

### Stable Integration of PiggyBac Plasmids

The following methods were used for stable integration of PiggyBac plasmids into iPSC.

Transfection of hiPSCs with the PiggyBac constructs was carried out as follows: iPSCs were dissociated into single cells using Accutase (Invitrogen) and replated at a density of 1.5×10⁶ cells in one well of a 6-well plate coated with Matrigel (Corning). The following day, 2 μg of PiggyBac construct pEXP-piB-BsD-Tet-NGN2-Puro-SNAP-PGKtk, 1.5 μg transposase pEf1α-hyPBase, and 10.5 μL TransIT-LT1 transfection reagent (Mirus) were added to 200 μL serum-free OPTI-MEM (Invitrogen). The transfection mix was incubated at room temperature for 20 min and added to cell culture containing 2 mL STEMFLEX medium that supports the robust expansion of feeder-free pluripotent stem cells (Invitrogen) supplemented with 10 μM ROCK inhibitor (Peprotech). After 6 hours incubation at 37° C. CO₂ incubator, the medium was changed to StemFlex plus 10 μM ROCK inhibitor. Media change was performed daily. On the second day of transfection, 5 g/mL blasticidin was added to 2 mL STEMFLEX plus 10 μM ROCK inhibitor. The media was changed every day. After five days blasticidin selection in the presence of ROCK inhibitor, cells were fed with StemFlex (no blasticidin or ROCK inhibitor) until the culture became confluent and ready for passaging and expansion of stably transfected cell line.

The following methods were used for transdifferentiation of iPSC to neurons. iPScs were lifted by incubating with ACCUTASE, a natural enzyme mixture with proteolytic and collagenolytic enzyme activity (Life Technologies), for 4 mins, combined with equal volume of STEMFLEX media, centrifuged at 800 rpm for 4 min, resuspended in STEMFLEX, and counted. Cells were seeded at a density of 1.25×10⁶ cells per well (for 6-well plates) with 0.5 μg/mL doxycycline to induce expression of the PiggyBac transgene and Ngn2. For 10-cm plates, 10 million cells were seeded. This was considered day 0. Plates were previously coated with Matrigel. For the first 2 days of neuron differentiation, media change was conducted daily with Neurobasal N2/B27 media (1×B27 supplement (Life Technologies), 1×N2 supplement (Life Technologies), 1×Non-Essential Amino Acids (Gibco), 1× GlutaMAX (Gibco), 1×Pen-Strep (Gibco), Neurobasal Media (Life Technologies)), 5 μg/mL blasticidin and 0.5 μg/mL doxycycline; for days 3-6, media changes were done the same as for days 1-2, with the addition of lug/mL puromycin to select cells expressing the PiggyBac transgene.

On day 7, ACCUTASE was used to dissociate the neurons before re-plating them onto the appropriate polyethyleneimine (PEI)/laminin-coated plates for downstream assays (e.g., 3 million cells per well of 6-well, 1 million cells per well of 24-well, 50,000 cells/well of 96-well plates). The following day (day 8), an equal volume of Neurobasal N2/B27 media supplemented with 20 ng/ml Brain-derived Neurotrophic Factor (BDNF; Peprotech 450-02), 20 ng/mL Glia-derived Neurotrophic Factors (GDNF; Peprotech 450-10), 1 mM Dibutryl cyclic AMP (cAMP; Sigma D0260), 2 μg/mL laminin, 1 μM AraC was added to the existing cell media. Doxycycline was withdrawn from medium on day 8. At day 11, media change occurred with equal volumes of Neurobasal N2/B27 and Neurobasal PLUS (Life Technology A35829-01) N2/B27 media, and 10 ng/ml BDNF, 10 ng/mL GDNF, 0.5 mM cAMP, 1 μg/mL laminin. At day 14, half media change occurred with Neurobasal Plus media, 10 ng/mL BDNF, 10 ng/mL GDNF, 1 mM cAMP, 1 μg/ml laminin. Half media change occurred every three days with Neurobasal Plus media, 10 ng/mL BDNF, 10 ng/mL GDNF, 0.5 mM cAMP, lug/mL laminin. For iPScs harboring PiggyBac-based doxycycline-inducible α-syn, 0.5 μg/mL doxycycline was kept in the media at every media change beyond day 7, whereas iPScs harboring PiggyBac-based doxycycline-inducible NGN2, doxycycline was not added to the media after day 7.

### Example 3: Rapid Induction of α-Syn Aggregates in iPSC-Derived Cortical Neurons

Transdifferentiation of α-syn triplication series iPSC (4-copy, 2-copy, 0-copy) into neurons was achieved as follows (FIG. 3A). A couple of days before starting trans-differentiation, iPSCs were cultured in STEMFLEX media with blasticidin (5 μg/mL) to ensure the lines in culture maintain the PiggyBac-Ngn2 construct. iPSC were dissociated into single cells using ACCUTASE (1 mL/well) and plated 1.5-2.0×10⁶ cells per well of a 6-well plate for transdifferentiation. On day 7, we proceeded with replating if the cells had acquired a neuronal morphology. If not, we continued changing the media on a 6-well plate until day 10. Once cells had acquired a neuronal morphology, neuronal cultures were reseeded using ACCUTASE for detachment and dissociation into single cells, and plated at 50K cells per well of a 96-well plate (100 μL per well).

Glass-bottom 96 square well plates (Brooks NC9662693) were coated with polyethylenimine (PEI) following the protocol for PEI coating, wrapped in parafilm and saran wrap, and stored at 4° C. overnight (0.1% PEI in Borate buffer). Polystyrene 96-well plates were plated with poly-l-lysine (PLL) or poly-l-ornithine (PLO) 20 μg/mL in PBS) O/N. The following day, the wells were washed three times with PBS and coated with laminin (5 μg/mL (Sigma-Aldrich) in PBS) with incubation for 2 hours at a 37° C., 5% CO2 incubator prior to plating the neurons.

On day 8, 100 μl of neurobasal media enriched with growth factors (BDNF, GDNF, cAMP) and AraC was added and doxycycline was withdrawn.

Every 3 days media was refreshed by adding 100 μl of Neurobasal media enriched with growth factors (BDNF, GDNF, CAMP).

On day 14, enough culture media was removed to leave 100 μL of culture media in the well (any extra media was discarded) and 100 μL of fresh neurobasal media enriched with growth factors (cAMP, BDNF, GDNF) and PFF (10 μg/mL in PBS) was added.

The media was refreshed by adding 100 μL of neurobasal media enriched with growth factors (BDNF, GDNF, CAMP) every 3 days.

On day 25, culture media was removed and 100 μl of fixative (4% PFA) was added for 15 min. Three washes with PBS were performed, waiting 5 min in between the washes, and continue with the immunofluorescence protocol.

Transdifferentiated neurons were exposed to synthetic α-syn PFFs (10 μg/mL) or brain-derived fibrils (10 μg/mL).

Synthetic α-syn PFFs were generated, first, by standard expression of the protein in competent E. Coli and purification by ion exchange followed by size exclusion chromatography. To generate synthetic pre-formed fibrils (PFFs), a 1 mg aliquot of lyophilized monomeric α-syn prepared in this way is resuspended in PBS, centrifuged for 10 min at 15000 g, transferred into a new tube under a sterile TC hood and an aliquot is being used to determine the concentration via absorbance at 280 nm using the Nanodrop One spectrophotometer. The solution is then diluted down to 5 mg/ml and incubated at 37° C. under shaking (1000 rpm) in a tabletop ThermoMixer equipped with a heated lid. After 7 days of incubation the aggregated PFF sample is aliquoted into appropriate volumes to prevent repeated freeze-thaw cycles, snap-frozen using a dry ice-ethanol bath and stored at −80° C. (Eppendorf LoBind tubes).

Pathologic α-syn (indicated by phosphorylation of Ser129; Abcam Ab51253) was observed in 4-copy, to a far lesser extent in 2-copy and not at all in 0-copy/knockout as shown in FIG. 3B. The neurons were also labeled with antibody to neuron-specific beta-III-tubulin and an AlexaFluor594 secondary antibody.

We generated brain-derived PFFs (schematized in FIG. 3C) using real-time quaking-induced conversion (RT-QuiC) as follows: 500 mg of frontal cortex from flash-frozen MSA and PD brain tissue was homogenized and subjected to serial extraction using detergents in increasing strength and subsequent ultracentrifugation to obtain an insoluble protein fraction containing aggregated α-syn as previously described (Peng et al. Nature 2018). For the RT-QuiC reaction (64, 65) used here to amplify and monitor α-syn aggregates, 10 μl of brain-derived seed was incubated with recombinant monomeric α-syn at 42° C. in a BMG FLUOstar Omega plate reader to amplify amyloid α-syn by incorporation of monomeric α-syn into the growing aggregate. Before each RT-QuiC experiment, lyophilized monomeric protein was dissolved in 40 mM phosphate buffer (pH=8), filtered using a 0.22 mm filter, and the concentration of recombinant protein was measured via absorbance at 280 nm using a Nanodrop One spectrophotometer. Brain-derived insoluble protein was tip-sonicated for 30 sec (1 sec off, 1 sec on) at 30% of amplitude and added to a 96 well plate with 230 mM NaCl, 0.4 mg/ml α-syn and a 3 mm glass bead (Millipore Sigma 1040150500). Repeated shaking (1 min incubation, 1 min double-orbital shaking at 400 rpm) disrupts the aggregates to produce an expanded population of converting units. The amyloid dye Thioflavin T was used in adjacent wells to monitor the increase of fibrillar content via fluorescence readings at 480 nm every 30 min until the signal plateaued towards the end of the amplification interval of six days.

Pathologic accumulation of α-syn was visualized for endogenous α-syn by probing with an antibody to α-syn pS129 (Abcam Ab51253) and an AlexaFluor488 secondary antibody. Neurons were labeled with antibody to neuron-specific β-III-tubulin (Biolegend 801201) and an AlexaFluor594 secondary antibody. Purity of glutamatergic cultures generated by transdifferentiation was confirmed with PCR to quantitate neuronal (MAP2), astrocyte (S100B) and lineage specific (TH/DA neuron, VGLUT1/cortical neuron) mRNAs. The results, shown in FIG. 3D, demonstrated pathologic accumulation of α-syn in the neurons harboring triplication of α-syn (4-copy) that were polymorphic and highly reminiscent of inclusions formed in postmortem brain from patients with matched disease.

### Example 4: All-In-One PiggyBac-Ngn2+ Toxic Protein Overexpression

To start investigating patient-specific pathology, we began with an iPSc line derived from a patient harboring an SNCA-A53T mutation (8); called “CORR” in FIG. 2). Briefly, an iPSc clone generated through mRNA-based reprogramming of this patient's fibroblasts was gene-corrected with CRISPR/Cas9-based editing to create an isogenic mutation-corrected clone. Briefly, a gRNA directed to the mutant site in exon 2 of SNCA was designed with an asymmetric repair donor template strategy as described in Richardson et al. (66).

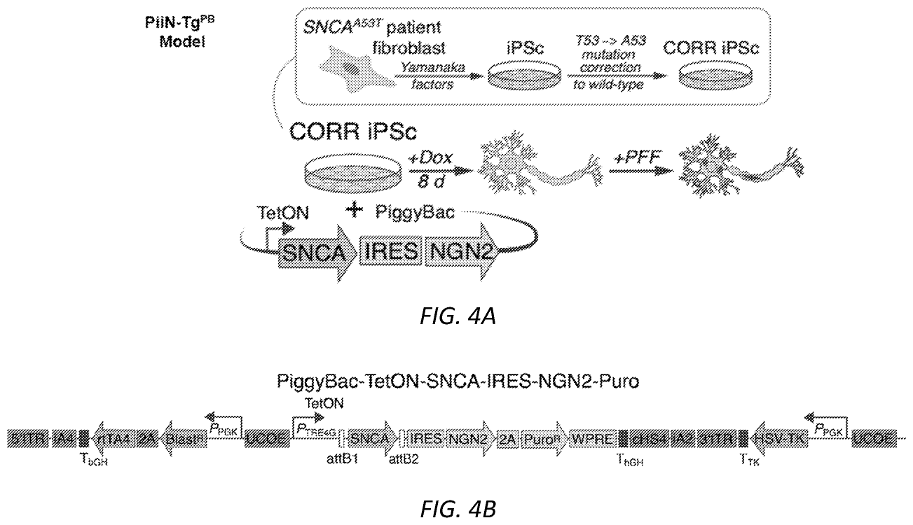

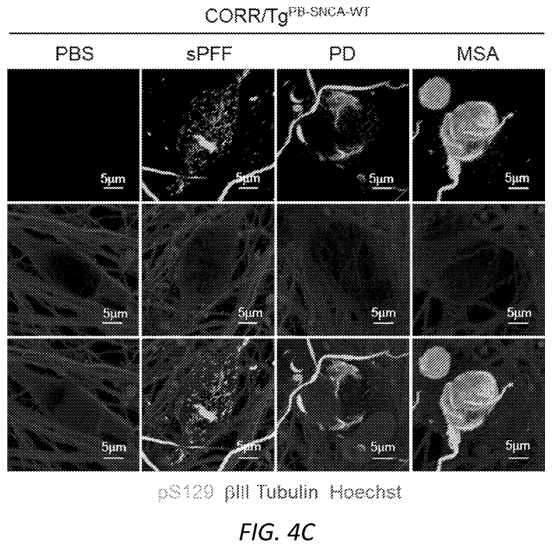

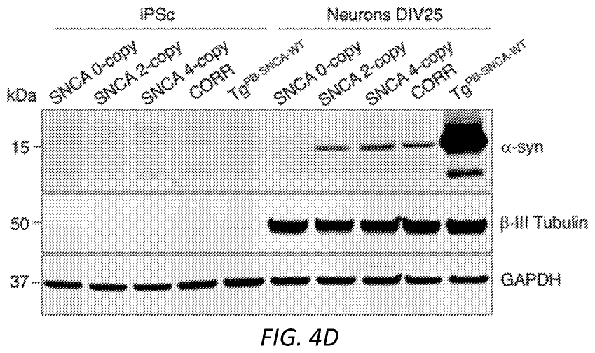

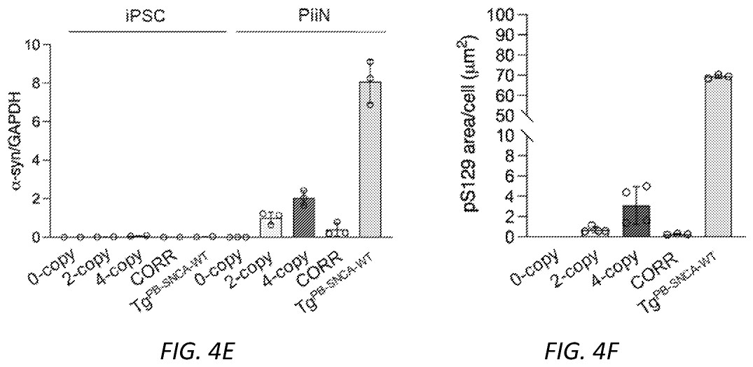

Into this mutation-corrected iPSC line, we introduced a bicistronic PiggyBac construct that co-expressed in a doxycycline-inducible manner WT α-syn in addition to the transcription factor neurogenin-2 (NGN2) for forced cortical neuron trans-differentiation (FIG. 4A-B). In more detail, the A53T-corrected hiPSc line (CORR-28) was subsequently modified by PiggyBac transposon-based integration of the TetO-SNCA-IRES-NGN2-Puro construct (and mutant versions and/or tagged derivatives). This line is henceforth referred to as CORR/Tg^{PB-SNCA-WT}. This transgenic construct (FIG. 4B) contains a Tet-On system for doxycycline-inducible expression of α-syn, followed by an internal ribosome entry site (IRES) sequence that allows direct transcription, and hence, co-expression of NGN2 from the same TetO promoter. Expression of the transcription factor Ngn2 allows direct differentiation of stem cells to cortical neurons (Zhang et al., 2013). The NGN2 sequence is followed by a puromycin resistance cassette that allows positive selection of cells expressing transgenic SNCA and NGN2 (thus, puromycin resistance occurs only after exposure to doxycycline). The PiggyBac-based TetO construct allows one-step (1) overexpression of α-syn, and (2) direct differentiation from iPSC to a nearly pure population of cortical neurons.

Transdifferentiation of transgenic α-syn-overexpressing neurons was performed as described in Example 2 and 3, except that doxycycline (500 ng/mL) was maintained throughout the protocol to maintain WT α-syn expression. Exposure of SNCA 0-copy, SNCA 2-copy, SNCA 4-copy neurons and CORR/Tg^{PB-SNCA-WT} to synthetic PFFs results in inclusion formation (FIG. 4C) and pathological accumulation of pS129-α-syn. CORR/Tg^{PB-SNCA-WT} neurons result in far higher α-syn expression (FIGS. 4D-E) and far more pathological α-syn inclusion formation (FIG. 4F). In this experiment, neurons were exposed to 10 μg/ml synthetic wild-type PFFs at Day 11 of differentiation and incubated for 14 days pre-analysis.

Whole cell extract and western blots were obtained as follows. For whole-cell lysis, iPSc-derived neurons were detached with 1×PBS. Neurons were transferred to a microcentrifuge tube and centrifuged for 5 min at 500 g and 4° C. The pellet was washed twice with 1×PBS, then extracted with 100 μL LDS buffer in the presence of protease and phosphatase inhibitors. The samples were tip-sonicated twice for 15 sec at 40% amplitude and centrifuged for 14 min at 15000 g at 4° C. The supernatant was collected, and protein concentration measured by BCA assay. Prior to SDS-PAGE, 30 μg of lysate was boiled at 65° C. for 5 minutes. Samples were loaded onto NuPAGE 4-12% Bis-Tris gel (Invitrogen). Gel electrophoresis was performed at 150V for 55 min in 1× NuPAGE MES Running Buffer (Invitrogen), and protein was transferred onto a Nitrocellulose membrane using the iBlot Gel Transfer Device (Invitrogen). The membrane was fixed in 4% paraformaldehyde for 30 min, washed three times with PBS for 5 min and blocked in Intercept Blocking Buffer (LiCor) for 1 hr at room temperature. The membrane was incubated in primary antibody in 5 0.1% Tween-20 Intercept Blocking Buffer overnight at 4° C. The next day, the membrane was washed 4 times with PBST (0.05% Tween-20), 5 min per wash, and incubated with IRDye fluorescent secondary antibodies (LiCor) for 1 hr. The membrane was subsequently washed 4 times with PBST (0.05% Tween-20), 5 min per wash, followed by one 5 min wash in PBS. All incubations were performed on a double-orbital shaker. The membrane was imaged using the LiCor Odyssey Lx imaging system. The primary antibodies used were against total α-syn (MJFR ab138501, 1:1000), GAPDH (Sigma G8796, 1:1000) and β-III Tubulin (Biolegend 801201, 1:2000).

### Example 5: Inclusion Formation in Both PiggyBac and STMN2 Transgenic α-Syn Lines is NAC Domain-Dependent

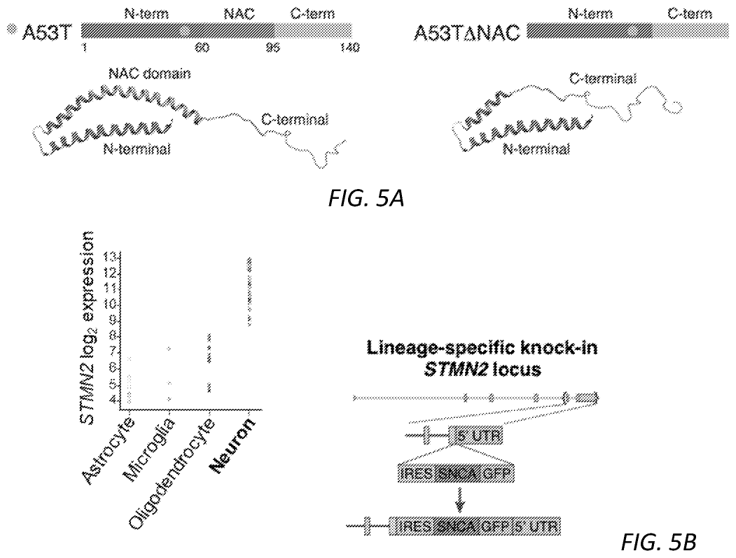

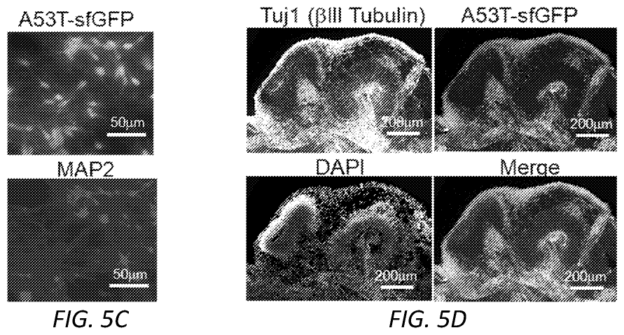

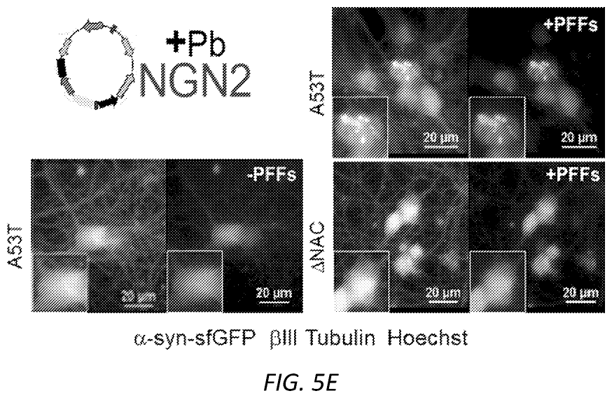

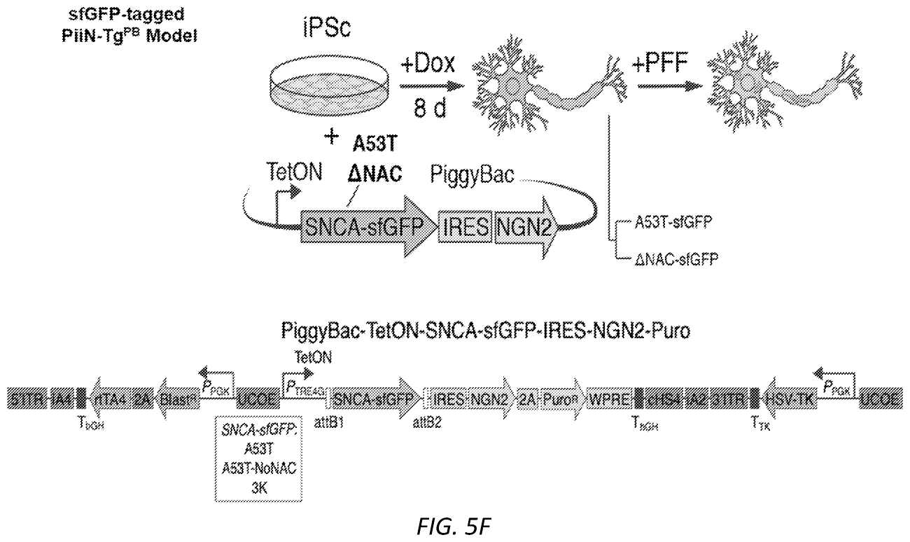

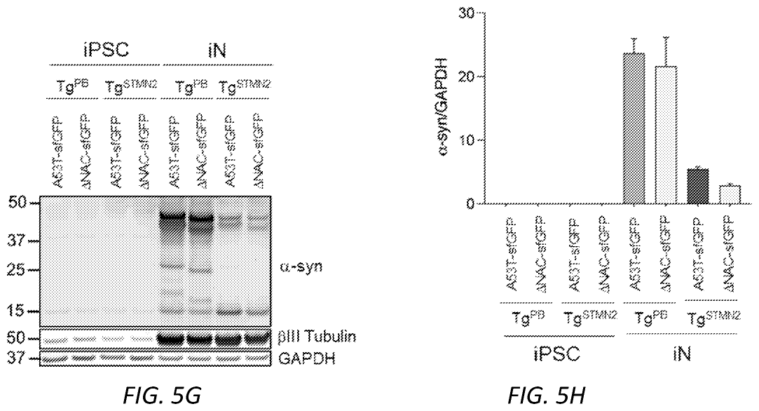

We developed an additional transgenic system in which transgenes were knocked in (utilizing a CRISPR/Cas9 strategy) to the 5′UTR of a relatively enriched in neurons (e.g., STMN2). This enables over-expression in a neuron-specific way that is dox-independent. In this transgenic model, α-syn (in this case A53T-sfGFP and A53T-ΔNAC-sfGFP as control; FIG. 5A) was targeted with CRISPR/Cas9 to a neuron-specific locus (STMN2) (H9 hESC). STMN2 expression is considerably higher in neuron than glia (scRNAseq data, human cortex; neuroexpresso.org; FIG. 5B). Targeting of α-syn expressing transgenes (in this case A53T-sfGFP and A53T-ΔNAC-sfGFP as control) by CRISPR/Cas9 to the STMN2 locus to enable neuron-specific expression of the transgene without use of doxycycline.

The following methods were used for STMN2 locus targeting in pluripotent stem cells (iPSc). This is captured schematically in FIG. 5B, lower. To generate the SNCA targeted STMN2 cell line, 750 bp homology arms were synthesized into custom gBlocks (IDT) and cloned plasmid FRT-PGK-Neo-TK to generate SMTN2-Targ-Neo. A transgene containing an IRES-SNCA or IRES-SNCAΔNAC coding sequence was then cloned into STMN2-Targ-Neo to generate the final targeting constructs, STMN2-Targ-Neo-SNCA or STMN2-Targ-Neo-ΔNAC. A 20 bp gRNA sequence which targeted the 3′ UTR of the STMN2 gene was cloned into plasmid pX330 to generate pX330-STMN2. For targeting the H9 cell line, the cells were accutased for 5 minutes at 37° C. and then briefly triturated to generate single cells. The single cell suspension was centrifuged at 200rcf for 5 minutes. The cell pellet was resuspended in DMEM/F12 and counted. 5 million cells were again centrifuged at 200rcf for 5 minutes. These cells were resuspended in 100 μL Lonza Stem Cell nucleofection solution from Human Stem Cell Nucleofector Kit 1. This cell mixture was then mixed with 1 microgram of plasmid pX330-STMN2 and 5 micrograms of STMN2-Targ-Neo-SNCA or STMN2-Targ-Neo-ΔNAC then nucleofected with program A-023. After nucleofection the cells were plated on 10-cm dishes with mTesr and ROCK inhibitor (10 μM) coated with Matrigel. 48 hrs after nucleofection the media was changed into mTesr with 100 μg/mL G418. The G418 selection was maintained for 10 days and then surviving colonies were manually picked into 96-well plates. To genotype the colonies the cells treated with EDTA (0.5 mM) for 5 minutes and then replica plated into another 96-well plate. After the plates had been grown to confluency, one of the plates was treated with 50 μl of QuickExtract buffer and lysed at 55° C. for 10 minutes. PCR primers pairs of which one bound outside of the targeting plasmids homology arm and the corresponding primer bound within the targeting plasmid were used to identify clones which harbored the correctly targeted SNCA or SNCAΔNAC transgenes. To identify additional random integration of the targeting plasmids, PCR primer pairs which amplified a sequence found in the backbone of the targeting plasmid were used with the correctly targeted clone lysates. The correctly targeted clones which did not harbor additional integrations of the targeting plasmid were expanded and cryopreserved.

The following methods were used for terminal neural differentiation after neural induction (FIG. 5C)
Day 1: Change media to 2 mLs BCG plus 2 μM PD0332991 and 10 μM DAPT
Day 3: Add 2 mLs BCG plus 2 μM PD0332991 and 10 μM DAPT
Day 5: Add 2 mLs BCG plus 2 μM PD0332991 and 10 μM DAPT
Day 7: Accutase cells and plate according to experimental design in BCG plus 2 μM PD0332991 and 10 μM DAPT and Rock Inhibitor.
Keep neurons in this media for 3 days then switch to just BCG media
BCG=Neurobasal base medium+N2/B27+BDNF (10 ng/ml), GDNF (10 ng/mL), dbCAMP (2 mM)+AA (0.4 μM)+laminin (2 μg/mL)

The following methods were used for transdifferentiation of knock-in Stmn2 lines to neurons (as shown in FIG. 5E). iPSC lines were differentiated into neurons by overexpression of the transcription factor neurogenin-2 (NGN2) as α-syn was being expressed. iPSc lines were engineered to express NGN2 under a tetracycline-inducible promoter. iPSc lines were dissociated and cultured (8500 cells per cm²) for 7 days in Neurobasal media enriched with B27 (50×), N2 (100×), NEAA (100×), Glutamax (100×), doxycycline (1 μg/mL), puromycin (1 μg/mL) and blasticidin (5 μg/mL). At 7 days in vitro (DIV), neuronal cultures were plated into a 96-well plate (156250 cells per cm²), previously coated with Poly-ethylenimine (0.1% PEI) and laminin (5 μg/mL), and exposed to growth factors (BDNF, GDNF and dibutyryl-cAMP). At 14 DIV, doxycycline was withdrawn and cortical neurons were exposed to synthetic preformed fibrils (PFF, 10 μg/mL) for two weeks. At 28 DIV, neuronal cultures are fixed (20% sucrose, 4% PFA in PBS), permeabilized, blocked (10% Donkey serum, 0.2% Triton X-100) and stained for neuron-specific β-III Tubulin.

The system functioned with conventional differentiation (FIG. 5C-D) or transdifferentiation (FIG. 5E).

The PiggyBac construct (bicistronic, expressing rtTA [generation 4] and Ngn2) enabled direct conversion from hESc into a ˜pure population of cortical-type neurons expressing α-syn constructs. In this case doxycycline was only used for transdifferentiation and then withdrawn. FIG. 5GE shows glutamatergic neurons transdifferentiated from the cells with dox-inducible Ngn2. Dox was then withdrawn and α-syn remains robustly expressed. One week exposure to synthetic pre-formed α-syn fibrils (PFFs) led to robust seeding and neuritic aggregates that was dependent on the NAC domain (FIG. 5E).

We generated forebrain organoids from this model to determine whether transgene expression continued through a complex differentiation. Day 30 organoids are shown in FIG. 5D.

An analogous system was developed to Example 4 except with over-expression of α-syn-A53T-sfGFP and α-syn-A53T-ΔNAC-sfGFP. This system thus expresses the same transgene targeted with the STMN2 expression system, except that the over-expression of α-syn constructs was achieved through random integration of the PiggyBac in the iPSC lines in a dox-dependent fashion. We found that the expression levels achieved with the PiggyBac transgenic system were far higher than with the transgenic STMN2 system.

### Example 6: PiggyBac Transgenic Reveals Distinct Fibrillar and Membrane-Rich α-Syn Inclusions Induced by PFFs

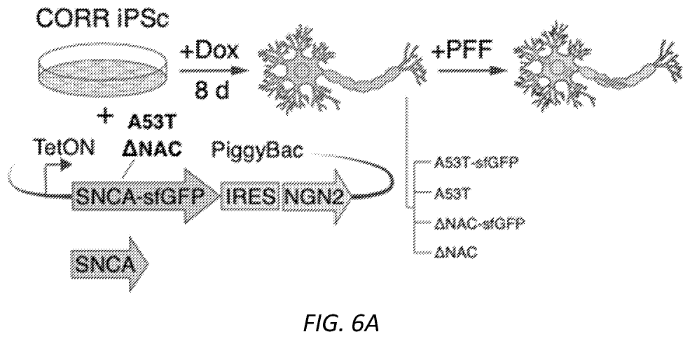

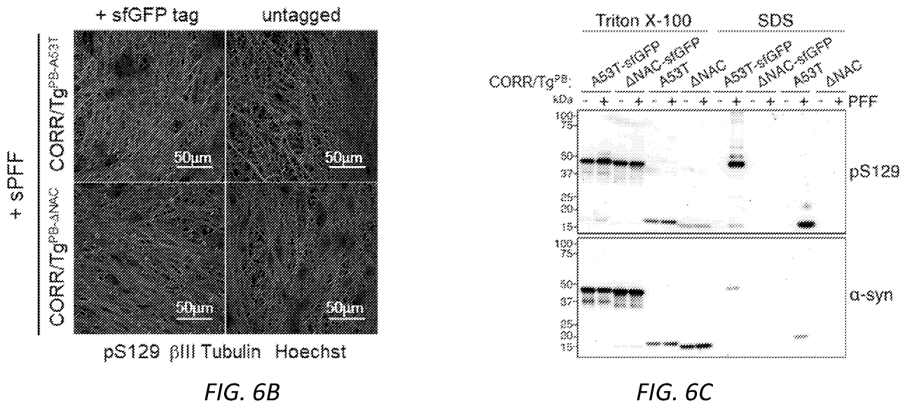

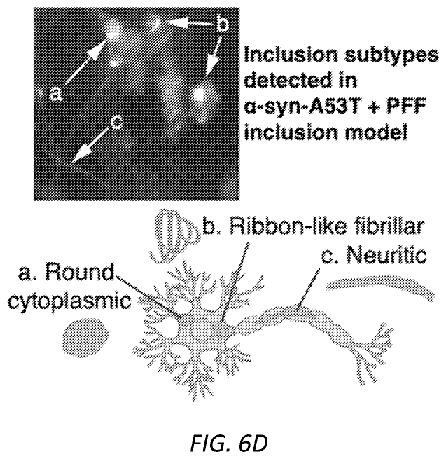

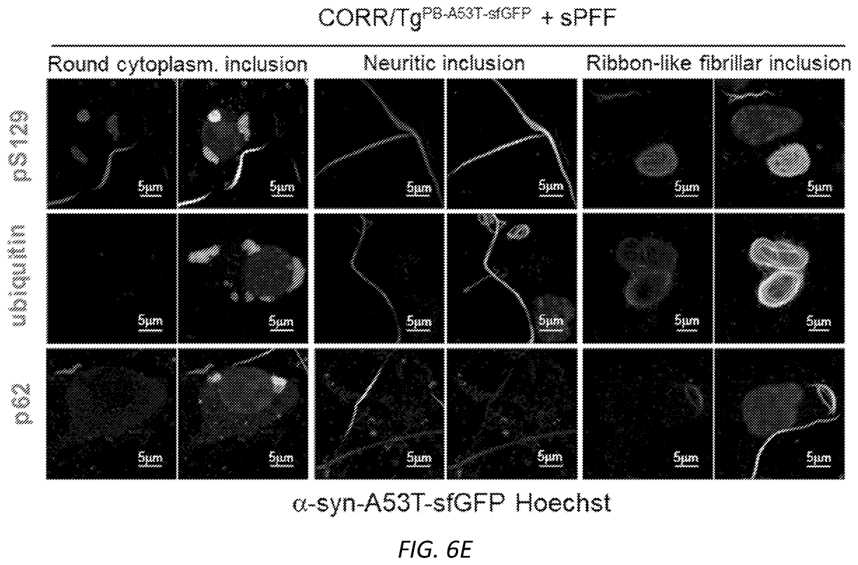

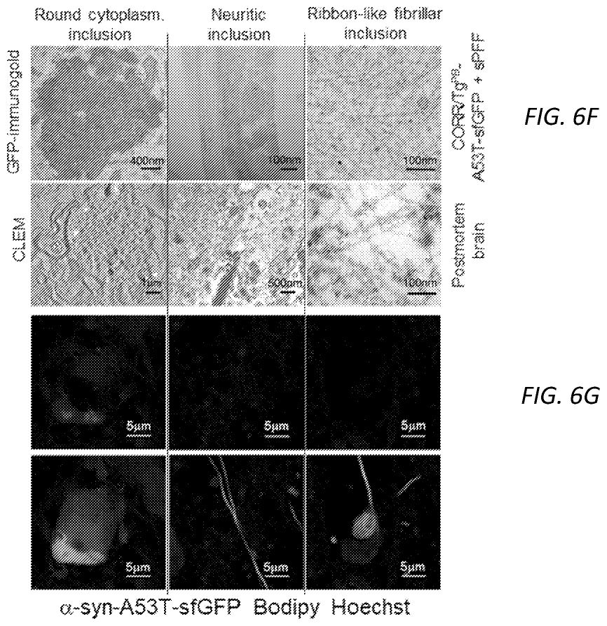

As illustrate in FIG. 6A, four PiggyBac constructs (based on plasmid #1022; Fig FIG. 5F) were stably introduced into the A53T mutation-corrected clone #28. Multiple constructs were individually introduced: α-syn-A53T; A53T-ΔNAC. Each of the α-syn constructs were expressed either untagged or with a C-terminal sfGFP tag.

Transfection of A53T-corrected hiPScs with the PiggyBac constructs was carried out as follows: iPScs were dissociated into single cells using Accutase (Invitrogen) and replated at a density of 1.5×10⁶ cells in one well of a 6-well plate coated with Matrigel (Corning). The following day, 2 μg of PiggyBac construct pEXP-piB-BsD-Tet-NGN2-Puro-SNAP-PGKtk, 1.5 μg transposase pEf1α-hyPBase, and 10.5 μL TransIT-LT1 transfection reagent (Mirus) were added to 200 μl serum-free OPTI-MEM (Invitrogen). The transfection mix was incubated at room temperature for 20 min and added to cell culture containing 2 mL StemFlex (Invitrogen) supplemented with 10 μM ROCK inhibitor (Peprotech). After 6 hours incubation at 37° C. CO₂ incubator, the medium was changed to StemFlex plus 10 μM ROCK inhibitor. Media change was performed daily. On the second day of transfection, 5 μg/mL blasticidin was added to 2 mL StemFlex plus 10 μM ROCK inhibitor. The media was changed every day. After five days blasticidin selection in the presence of ROCK inhibitor, cells were fed with StemFlex (no blasticidin or ROCK inhibitor) until the culture became confluent and ready for passaging and expansion of stably transfected cell line.

For routine passaging and expansion, stem cells were washed with 1 mL 1 mM EDTA in PBS, then incubated with 1 mL EDTA (1 mM) in PBS for 4 minutes at room temperature. The EDTA solution was aspirated, and cells were harvested from the well with 1 mL StemFlex, transferred to a 15 mL Falcon tube, centrifuged for 3 minutes at 800 rpm, resuspended in an appropriate volume of StemFlex, and distributed at the desired ratio (e.g., from 1 well onto 3 wells) to a new Matrigel-coated plate.

NGN2-Induced Neuron Differentiation iPScs were lifted by incubating with Accutase (Life Technologies) for 4 min, combined with equal volume of StemFlex media, centrifuged at 800 rpm for 4 min, resuspended in StemFlex, and counted. Cells were seeded at a density of 1.25×10⁶ cells per well (for 6-well plates) with 0.5 μg/mL doxycycline to induce expression of the PiggyBac transgene and Ngn2. For 10-cm plates, 10 million cells were seeded. This was considered day 0. Plates were previously coated with Matrigel. For the first 2 days of neuron differentiation, media change was conducted daily with Neurobasal N2/B27 media (1×B27 supplement (Life Technologies), 1×N2 supplement (Life Technologies), 1× Non-Essential Amino Acids (Gibco), 1× GlutaMAX (Gibco), 1× Pen-Strep (Gibco), Neurobasal Media (Life Technologies)), 5 μg/mL blasticidin and 0.5 μg/mL doxycycline; for days 3-6, media changes were done the same as for days 1-2, with the addition of 1 μg/mL puromycin to select cells expressing the PiggyBac transgene.

On day 7, Accutase was used to dissociate the neurons before re-plating them onto the appropriate polyethyleneimine (PEI)/laminin-coated plates for downstream assays (e.g., 3 million cells per well of 6-well, 1 million cells per well of 24-well, 50,000 cells/well of 96-well plates). The following day (day 8), an equal volume of Neurobasal N2/B27 media supplemented with 20 ng/mL Brain-derived Neurotrophic Factor (BDNF; Peprotech 450-02), 20 ng/ml Glia-derived Neurotrophic Factors (GDNF; Peprotech 450-10), 2 mM Dibutyryl cyclic AMP (cAMP; Sigma, D0260), 2 μg/mL laminin, 0.5 μM AraC was added to the existing cell media. At day 11, media change occurred with equal volumes of Neurobasal N2/B27 and Neurobasal PLUS (Life Technology A35829-01) N2/B27 media, and 10 ng/ml BDNF, 10 ng/ml GDNF, 1 mM CAMP, 1 μg/mL laminin. At day 14, half media change occurred with Neurobasal Plus media, 10 ng/mL BDNF, 10 ng/mL GDNF, 1 mM cAMP, lug/mL laminin. Half media change occurred every three days with Neurobasal Plus media, 10 ng/ml BDNF, 10 ng/ml GDNF, 1 mM cAMP, 1 μg/mL laminin. For iPScs harboring PiggyBac-based doxycycline-inducible α-syn, 0.5 μg/mL doxycycline was kept in the media at every media change beyond day 7, whereas iPScs harboring PiggyBac-based doxycycline-inducible NGN2, doxycycline was not added to the media after day 7.

### Induced Astrocyte Differentiation

On day 0, H1 hESCs at ˜95% confluency were dissociated with Accutase, and 4×10⁶ cells were replated in Matrigel-coated 10-cm dishes using StemFlex medium with 10 μM ROCK inhibitor (StemCell Technologies, Y-27632) and 500 ng/mL doxycycline. On days 1 and 2, cells were cultured in Expansion medium (DMEM/F-12, 10% FBS, 1% N2 supplement, 1% Glutamax (Thermo Fisher Scientific)). From days 3 to day 5, Expansion medium was gradually switched to FGF medium (Neurobasal, 2% B27 supplement, 1% NEAA, 1% Glutamax, and 1% FBS (Thermo Fisher Scientific); 8 ng/mL FGF, 5 ng/ml CNTF, and 10 ng/mL BMP4 (Peprotech)). On day 6, the mixed medium was replaced by FGF medium. Selection was carried out on days 1-6 with 5 μg/mL blasticidin for cell lines harboring vectors conferring blasticidin resistance. On day 7, cells were dissociated with Accutase and replated in Matrigel-coated wells. The day after, FGF medium was replaced, and afterwards 50% of the medium was replaced by Maturation medium (1:1 DMEM/F-12 and Neurobasal, 1% N2, 1% sodium pyruvate, and 1% Glutamax (Thermo Fisher Scientific); 5 mg/mL N-acetyl-cysteine, 500 mg/mL dbcAMP (Sigma-Aldrich); 5 ng/ml heparin-binding EGF-like growth factor, 10 ng/mL CNTF, 10 ng/mL BMP4 (Peprotech)) every 2-3 d, and cells were kept for either 8 days or 21 days.

### PEI/Laminin Coating

96-well plates (MatriPlate MGB096-1-2-L-G-L) were coated with polyethyleneimine (PEI) and laminin for day 7 passaging of the iPSc-derived neurons. 800 μL 5% PEI was diluted in 40 mL 2× borate buffer to make 0.1% PEI and filter sterilized. 150 μL of the PEI/borate buffer solution was added to each well of the 96-well plate. The plate was wrapped in parafilm and saran wrap, and stored at 4° C. overnight. On the morning of passaging, each PEI-coated well from the 96-well plate was washed twice with 300 μL of water and once with PBS. Each well was coated with 150 μL of 5 μg/mL laminin (L2020 Sigma) in PBS and the plate was incubated for at least 2 hours at 37° C. prior to seeding the cells.

Immunofluorescence analysis was performed as follows. iPSc-derived neuron cultures were fixed with 100 μL of 4% paraformaldehyde, 20% sucrose in PBS. Cells were blocked and permeabilized in 10% goat serum, 0.2% Triton X-100 in PBS for 1 hour at room temperature. Primary antibody was incubated in 2% goat serum, 0.04% Triton X-100 overnight at 4° C. Cells were washed three times with PBS, 5 min per wash, and incubated with secondary antibody in 2% goat serum, 0.04% Triton X-100 and 0.1% Hoechst for 1 hour at 37° C. Finally, cells were washed three times with PBS, 5 min per wash. For immunostaining of E3xK-sfGFP and sfGFP iPSc-derived neurons, saponin was used instead of Triton X-100 since this detergent was found to preserve inclusions better than Triton X-100 when immunostaining. Images of the immunostained cells were captured with a Nikon TiE/C2 fluorescence microscope.

Triton/SDS sequential extraction of sfGFP-tagged lines was performed as follows. Sequential extraction with Triton X-100 and SDS was performed as described in Volpicelli-Daley et al. (50). Briefly, neurons that were seeded at 3×10⁶ cells/well in 6-well plate were rinsed twice with PBS, kept on ice, and scraped in the presence of 250 μl of 1% (vol/vol) Triton X-100/TBS with protease and phosphatase inhibitors. The lysate was transferred to polyallomar ultracentrifuge tubes and sonicated ten times at 0.5 s pulse and 10% power (Misonix Sonicator S-4000). Samples were incubated on ice for 30 min, then centrifuged at 100,000 g at 4° C. for 30 min in an ultracentrifuge. The supernatant (Triton X-100 extract) was transferred to a microcentrifuge tube and combined with 4× Laemmli buffer for SDS-PAGE (small aliquot of ˜20 μL is saved prior to mixing with Laemmli buffer for protein assay. In the meantime, 250 μL of 1% Triton X-100/TBS was added to the pellet and sonicated ten times at 0.5 s pulse and 10% power, followed by ultracentrifugation at 100,000 g at 4° C. for 30 min. Next, 125 μL of 2% (wt/vol) SDS/TBS with protease and phosphatase inhibitors was added to the pellet. The sample was sonicated fifteen times at 0.5 s pulse and 10% power, ensuring that the pellet is completely dispersed. The supernatant (SDS extract) was transferred to a new microcentrifuge tube and diluted to 2× volume for the corresponding Triton X-100 fraction to make the insoluble α-syn species more abundant and easier to visualize by western blot. For example, 60 μL of 4× Laemmli buffer was added to 180 μL of Triton X-100 extract, and 30 μL of 4× Laemmli buffer to 90 μL SDS extract.

BCA protein assay was performed on the Triton X-100 supernatant and SDS extract. For SDS-PAGE, 5 μg of protein samples were boiled for 5 min, centrifuged for 2 min at maximum speed, and loaded onto 4-12% Bis-Tris gel. The samples were electrophoresed at 150V for approximately 90 min. Protein was transferred to PVDF membrane using iBlot 2 Dry Blotting System (Invitrogen). The membrane was fixed for 30 min in 0.4% PFA/PBS if detecting untagged α-syn. The membrane was subsequently blocked for 1 h with 5% (wt/vol) milk/TBS before incubating with primary antibody overnight at 4° C. with shaking. The primary antibody was diluted in 5% (wt/vol) milk/TBS. The following primary antibodies were used: rabbit anti-PS129 (Abcam 51253) 1:5000, rat anti-α-syn 15G7 (generously provided by Ulf Dettmer) 1:300, goat anti-GFP (Rockland 600-101-215) 1:5000, mouse anti-GAPDH (Thermo Fisher MA5-15738) 1:5000. After incubation with primary antibody, the membrane was rinsed three times with TBS/T, 10 min with rocking for each rinse. The membrane was then incubated with HRP-conjugated secondary antibodies for 1 h at room temperature, with rocking. The following secondary antibodies were used: anti-rat-HRP (Sigma Aldrich NA935) 1:10,000, anti-rabbit-HRP 1:10,000 (Bio-Rad 170-6515), anti-goat-HRP 1:10,000 (R&D Systems HAF109). The membrane was rinsed three times with TBST/T, 10 min per rinse, with rocking, before developing with chemiluminescence.

Electron microscopy (EM) was performed as follows. iPSc-derived neurons were seeded at day 7 at either 3 million cells/well in poly-L-ornithine (PLO)/laminin pre-coated 6-well plate (Corning) (E3xK-sfGFP experiment), or 0.3 million cells on Aclar coverslips coated with PEI/laminin (A53T-sfGFP experiment). At 3-4 weeks of differentiation, iPSc-derived neurons were fixed in 2.5% glutaraldehyde, 1.25% paraformaldehyde, 0.03% picric acid in 0.1 M sodium cacodylate buffer (pH 7.4) for one hour at room temperature. Fixed neurons were brought to the Harvard Medical School Electron Microscopy Facility, where they were processed by washing three times with 0.1M Cacodylate buffer, incubating in 1% Osmium tetroxide (OsO4)/1.5% potassium ferrocyanide (KFeCN6) for 30 minutes, followed by a series of three washes and 30 minutes incubation in 1% aqueous uranyl acetate. Water was used to wash the neurons twice and they were dehydrated in 50%, 70%, 95% and twice in 100% alcohol. Neurons were embedded at 60° C. for two days in TAAB Epon. Approximately 80 nm sections were made on a Reichert Ultracut-S microtome and deposited up on to copper grid. These sections were immunogold labeled by etching with a saturated solution of sodium metaperiodate in water for five min at room temperature. The grids were washed trice in water and floated on 0.1% Triton-X-100 for 5 minutes at room temperature. 1% BSA+0.1% TX-100/PBS was used to block the grid for an hour at room temperature. Incubation with anti-GFP antibody (1:50, Abcam 6556) in 1% BSA+0.1% TX-100/PBS overnight at 4° C. was performed. Grids were washed three times in PBS to remove unbound GFP antibody and incubated with 15 nm Protein A-gold particles (Department of cell biology, University Medical Center Utrecht, the Netherlands) for 1 hour at room temperature. PBS and water were used to wash the grids and lead citrate was used to stain them. The grids were examined in a JEOL 1200EX Transmission electron microscope (JEOL USA Inc. Peabody, MA USA). The electron micrographs were recorded with an AMT 2k CCD camera.

### Example 7: E3xK Inclusion Formation Recapitulates Lipid-Rich Inclusions Found in PFF-Seeded Neurons

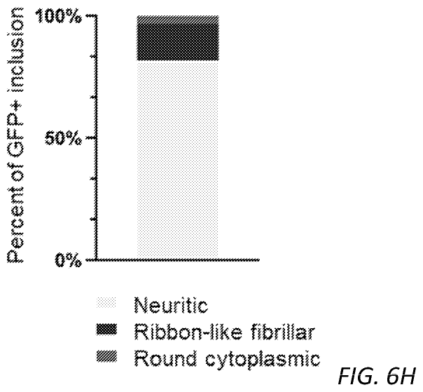

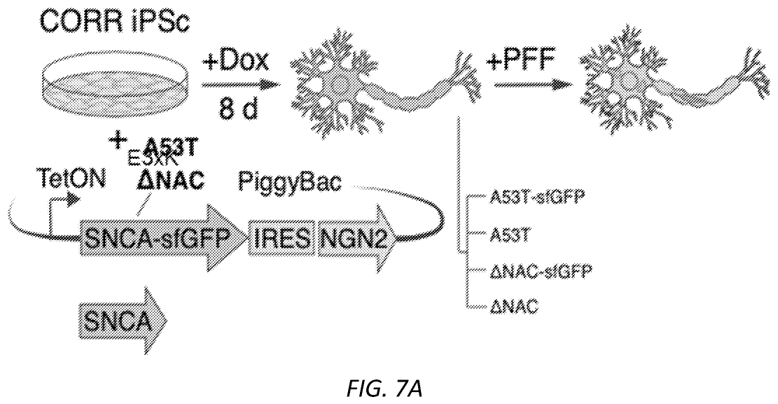

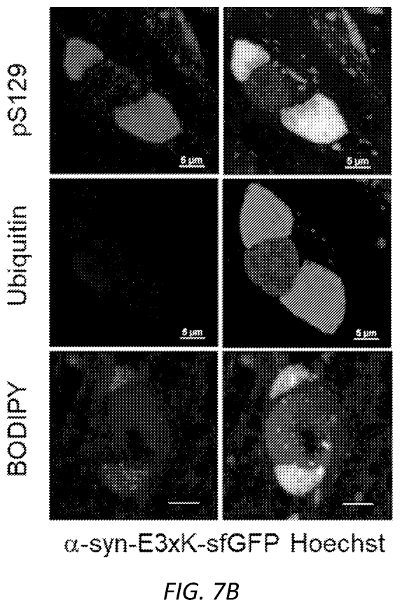

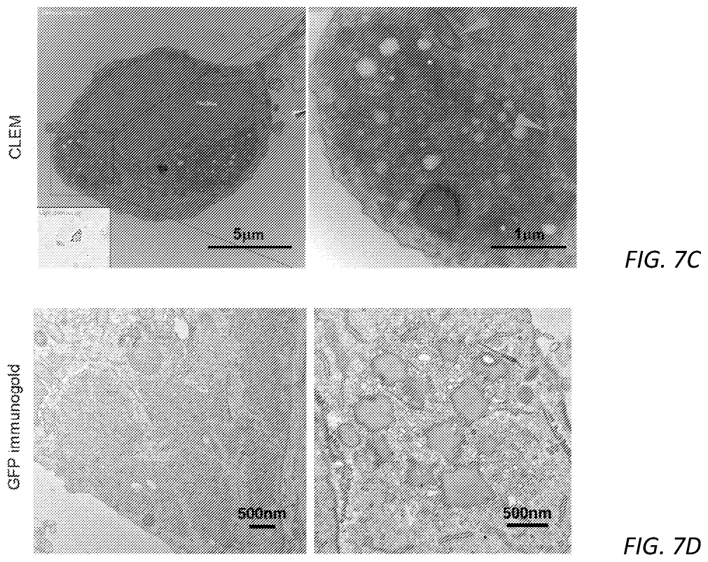

The pathogenic α-syn mutation (E46→K) enhances membrane affinity, an effect amplified by additional E→K mutations (E3xK: E35K+E46K+E61K). Motivated by a recent mouse model expressing E3xK (that exhibits robust cortical and DA neuron loss, PB/LB pathology and a levodopa-responsive tremor (21)) we developed an analogous model in human neurons. The A53T mutation-corrected familial synucleinopathy line mentioned previously was used to generate PiggyBac transgenic lines that, upon addition of doxycycline, simultaneously trans-differentiated into cortical neurons and expressed untagged or sfGFP-tagged α-syn E3xK. Inclusions formed in E3xK lines but not sfGFP control. Inclusions were strongly immunopositive for α-syn pS129 (Abcam, ab51253) (FIG. 7B) but not ubiquitin (Millipore Sigma, clone FK2), another hallmark feature of classical Lewy bodies. Staining for neutral lipids using a BODIPY dye showed that these inclusions are lipid-rich in content, analogous to the round dynamic inclusions in the A53T+PFF model described previously. Inclusions also colocalized with early (Rab5), late (Rab7) and recycling (Rab11) endosomal markers (20). CLEM of E3xK-sfGFP in iPSc neurons showed a membranous medley ringed by mitochondria (FIG. 7C) that was strikingly similar to PBs of postmortem PDD brain (Braak Stage V; from Dr. Henning Stahlberg). Correlative light and electron microscopy was performed by Dr. Amanda Lewis (Stahlberg lab, EPFL, Switzerland). Neurons were fixed in 0.1M cacodylate buffer, 4% EM grade paraformaldehyde, and 0.1% glutaraldehyde in water. Processing of coverslips and resin embedding was performed as described, and resin-embedded samples were mounted onto glass slides and immunostained as described in Shahmoradian et al. (9), and using the full-length α-syn antibody LB509. Light microscopy images were taken on a Nikon Ti-E widefield microscope. Immediately adjacent tissue sections mounted onto electron microscopy grids were imaged on a Talos 200 keV transmission electron microscope.

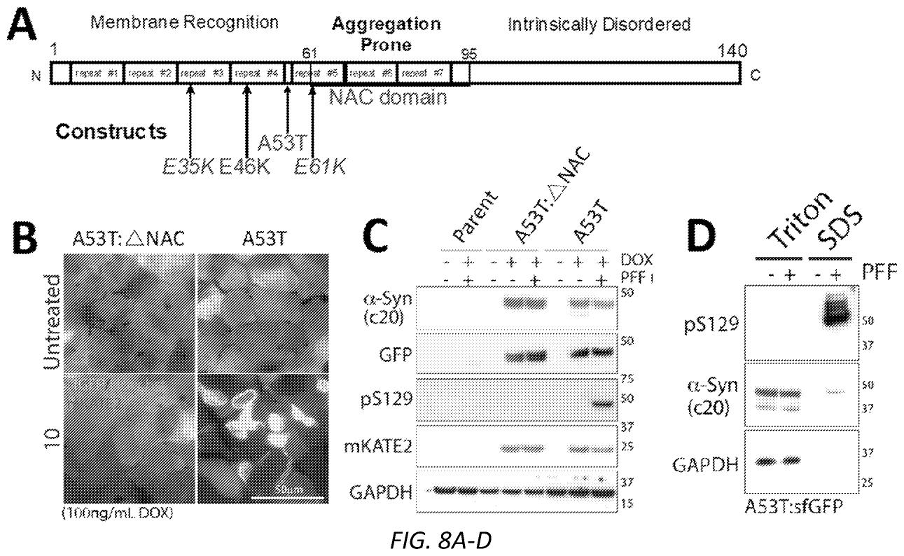

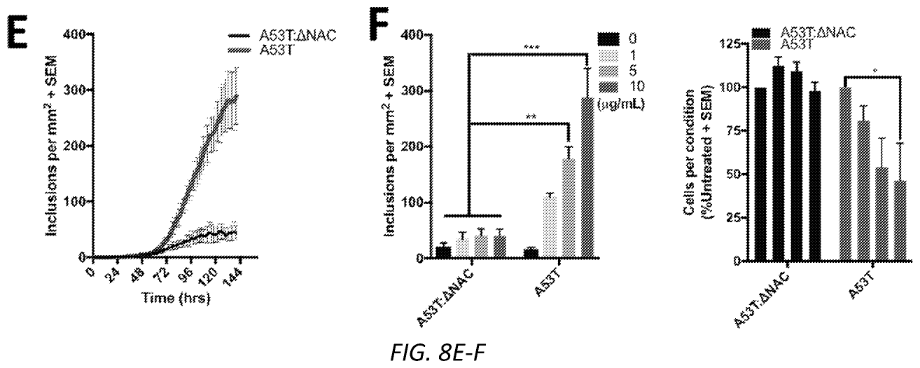

We also developed an analogous model for clustered vesicle inclusions. We expressed the E3xK mutation in U2OS cells in the same inducible system (FIGS. 8G-J). Exactly analogous to its expression in neuronal cell lines and primary neurons (whether untagged or fused to EYFP (20, 67), E3xK in U2OS cells recapitulated key features of pathological α-syn, including formation of round inclusions (FIG. 8G) and toxicity (FIG. 8I). The inclusions began within 10 hrs after dox induction (FIG. 81) and increased in size over time, likely due to fusion events (20). Unlike the PFF-model, the 3K inclusions appeared to be NAC-independent (FIG. 8J), indicating a fundamentally different biology to the amyloid seeding model. Microscopically, these non-fibrillar inclusions were a medley of α-syn and vesicles of various sizes, positive for lysosomal markers such as LAMP-1, endosomal Transferrin Receptor, Golgi (SitN15) and other vesicle markers (20). Association with lipid droplets and indications of membrane tubulation within the α-syn/vesicle clusters indicated severe perturbations of membrane and lipid homeostasis (20).

### Example 8: Unbiased Genome-Wide Screen for Disease Process Modifiers in a Simple Cellular Model

We developed a tractable model in which human U2OS cells (which are ideally suited for microscopy) were seeded with synthetic and brain-derived fibrils (FIGS. 8A-J). Inducible expression (using a bicistronic piggybac construct harboring reverse tetracycline transactivator, as shown in FIG. 1B) of fluorescently (sfGFP) tagged α-syn enabled visualization of α-syn in living cells. The system was fully GATEWAY-compatible allowing versatile expression of different α-syn mutants. When seeded with synthetic PFFs (50), intracellular inclusions formed aggregated α-syn in postmortem brain and were triton-insoluble (FIG. 8D), ubiquitinated, phosphorylated at Ser129 (FIG. 8D) and dependent on host-cell expression of the “non-amyloid B-component” (NAC) domain of α-syn (FIGS. 8A-F).

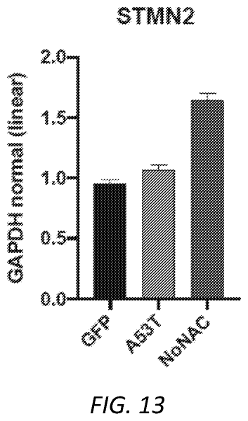

We developed an unbiased screen design, an illustration of which is provided in FIG. 9. This assay used a pooled screen format, and the assay was survival. We tested this assay in the U2OS model. FIG. 9 shows pooled Crispr/Cas9-based screens that covered most genes of the genome with 5 guide RNAs per gene. We completed screens for α-syn, in this case with an amplified E→K mutation (E3xK) compared to an sfGFP control. We completed two CRISPR screens now (wild-type α-syn versus E3xK synuclein versus sfGFP; A53T α-syn versus A53T-ΔNAC-α-syn versus sfGFP). To confirm that the CRISPR gRNA library and the screen performed well, we examined whether gRNAs targeting essential genes were depleted. FIG. 13 provides the fold-change in read counts from day 14/day 0 in each of the 3 lines. Essential genes were depleted in all 3 lines. Fold-change (read counts d14/d0) of gRNA targeting essential genes shows depletion, so the results confirmed that the CRISPR gRNA library and the screen performed well.

The CRISPR screen was conducted as follows: cell lines were expanded to 27×15-cm plates/line at 10 million cells/plate for a total of 270 million cells for the start of the screen. U2OS cells were passaged by washing adherent cells with 1×DPBS, incubating with Trypsin for 5 min at 37° C., centrifuging for 5 min at 300 g, aspirating the supernatant, resuspending the cell pellet in growth media (McCoy's 5A, 10% fetal bovine serum (FBS), penicillin-streptomycin), and plating at the desired cell density. Cells were infected with a gRNA/Cas9 lentivirus library at low MOI (0.2) with a representation of 500 cells/gRNA in triplicate (for three replicate screens), followed by puromycin (2 μg/mL) selection for 1 week, or until an uninfected control plate completely died. An initial cell pellet (50 million cells) was harvested as day 0 after expansion of the puromycin-selected cells to the appropriate scale to begin the screen (100 million cells/line). The remaining 50 million cells were re-plated and treated with doxycycline (100 ng/mL) to induce α-syn. Cell pellets were harvested 7 days and 14 days after doxycycline induction. Genomic DNA was isolated from the day 0, 7, 14 cell pellets by phenol: chloroform extraction. Briefly, the cell pellet is resuspended in TE (10 mM Tris pH 8.0, 10 mM EDTA) to a final concentration of 2-10 million cells/mL of TE, and combined with 0.5% SDS and 0.5 mg/mL Proteinase K. The suspension is incubated at 55° C. overnight, with shaking/inverting the cell suspension over the course of one hour to ensure complete digestion. Next, 0.2M NaCl is added, followed by phenol chloroform extraction in phase lock gel tubes. Equal parts of phenol: chloroform and sample are mixed in phase lock gel tubes, shaken for 1 minute to extract, then centrifuged for 5 minutes. The DNA aqueous phase will be the top layer, which is subsequently chloroform extracted by mixing equal parts with chloroform, shaken for 1 minute, and centrifuged for 5 min. The tubes are incubated with the caps open for 1 hour at 50° C. to evaporate the chloroform. Samples are treated with 25 μg/mL RNase A overnight at 37° C., then extracted with phenol: chloroform and chloroform as described above. DNA is precipated with ethanol overnight at −20° C., or for 3 hours at −80° C. Next, 1/10 v/v 3M sodium acetate pH 5.2 and 2 volumes 100% ethanol are added and the mixture centrifuged for 30-45 min at 4500 rpm at 4° C. The DNA pellet is washed once with 70% ethanol and transferred to an Eppendorf tube, followed by two more washes with 70% ethanol. The DNA pellet is dried at 37° C. for 10-20 min, then resuspended in 1 mL EB/TE by incubating at 55° C. gRNAs were PCR amplified with barcoded primers for sequencing on an Illumina NextSeq 500. Sequencing reads were aligned to the initial library and counts were obtained for each gRNA.

To determine the amount of virus to use in the infection, a titering experiment was performed in which 10-fold serial dilutions of the virus (10 μL, 1 μL, 0.1 μL, 0.01 μL, 0.001 μL) were used to infect cells seeded in a 6-well plate at the same seeding density as a 15-cm plate (i.e., 17-fold fewer cells based on the surface area difference between a 6-well plate and a 15-cm plate). Growth media supplemented with 2 μg/mL puromycin was added 1 day after infection and selection proceeded until the uninfected well was completely dead. The amount of virus resulting in 60-80% killing was recorded. This virus amount translates to a multiplicity of infection (MOI) of around 0.2-0.3 for the screen.

The number of cells needed for the start of the screen depends on the size of the library to be screened. For a library of 40,000 gRNAs and a representation of 500 cells/gRNA, 20 million cells are required per replicate. For screening in triplicate, this means that 60 million cells are required. A low MOI is used to ensure that there is only 1 gRNA per cell, thus 3-5 times as many cells as virus are required. Taken together, a library of 40,000 gRNAs at a representation of 500 in triplicate requires 180-300 million cells at the start of the screen.

### Example 9: Generation of Targeted Inducible Transgene at AAVS1 Locus in hESC Via TALENs

To establish a Tet-On system transgene at the AAVS1 locus within the PPP1R12C gene, two rounds of TALEN-mediated gene editing were conducted in hESC lines (WIBR-1 clone 22 or WIBR-3 clone 38). First, one construct containing the M2rtTA reverse tetracycline transactivator under the control of the constitutive CAGGS promoter (P_{CAGGS}-M2rtTA) was targeted to one AAVS1 allele. The second AAVS1 allele was subsequently targeted with a construct containing the transgene of interest driven by the M2rtTA-responsive TRE-Tight promoter (e.g., P_{TRE-Tight}-SNCA-mK2). Both constructs have flanking 5′ AAVS1 and 3′ AAVS1 homology arms.

Integration of the Tet-On constructs at the AAVS1 locus was confirmed by Southern blot analysis. Genomic DNA was extracted from a well of a 12-well plate at 70-90% confluency according to the manufacturer's manual (DNeasy Blood and Tissue Kit, Qiagen), and digested with EcoRV-HF (NEB) restriction enzyme. DNA restriction fragments were size-fractionated by electrophoresis in a 0.8% agarose gel, washed for 15 min in 0.25 M HCl solution (nicking buffer) at 80 rpm, followed by 15 min at 80 rpm in 0.4 M NaOH solution (denaturing and transfer buffer), and assembled in a transfer stack for alkaline Southern transfer onto a nylon membrane. The transfer membrane was rinsed in 0.2 M Tris-Cl, pH 7.0 and subsequently in 2×SSC (0.3 M NaCl/7.5 mM trisodium citrate) for 2 min each at 80 rpm. The transfer membrane was dried in a 55 C oven for 15 min, followed by pre-hybridization step with hybridization buffer (1% (w/v) BSA, 1 mM EDTA, 0.5M NaPO₄, 7% (w/v) SDS) in a 60 C hybridization oven for 1 h with rotation. Radioactive labeling of AAVS1 internal 5′ probe corresponding to the 5′ homology arm of the AAVS1 donor targeting vector was carried out by random-sequence oligonucleotide-primed DNA synthesis in the presence of the Klenow fragment of the E. coli DNA polymerase I, a 3dNTP mix (minus dCTP) and the radioactively labeled nucleotide [α-³²P]dCTP for 30 min at 37 C. The radiolabeled probe DNA was separated from unincorporated dNTPs by gel filtration chromatography using pre-equilibrated CHROMA SPIN columns (Clontech) with centrifugation at 3,500 rpm for 5 min. The double-stranded probe DNA was denatured at 100° C. for 5 min prior to adding to fresh hybridization buffer and hybridizing overnight in the 60° C. hybridization oven with rotation. After the hybridization step, the DNA blot was washed in 2×SSC (0.3 M NaCl/7.5 mM trisodium citrate/0.2% (w/v) SDS (low-stringency wash) for 30 min in a gently shaking 60 C water bath. Any remaining nonspecifically bound probe DNA was washed off during a high-stringency wash with 0.2×SSC (0.03 M NaCl/0.75 mM trisodium citrate)/0.2% (w/v) SDS) in a 60 C water bath with gentle shaking for a minimum of 20 min. The membrane was then sealed in Saran wrap, placed between an autoradiography film and an intensifying screen, exposed for 24-72 h at −80 C, brought to room temperature, and developed using the Kodak X-OMAT 1000A film processor.

To re-hybridize the DNA blot with an AAVS1 external 3′ probe which hybridizes with a sequence downstream of exon 3 of the PPP1R12C gene, the transfer membrane was rinsed in 0.08 M NaOH solution (stripping buffer) at room temperature with gentle shaking for a minimum of 15 min. The transfer membrane was subsequently washed three times with 2×SSC for 5 min each. If any radioactive signal was still detectable, the nylon membrane was stripped in 0.4 M NaOH (denaturing and transfer buffer) for 30 min at room temperature, with gentle shaking. The transfer membrane was dried between two Whatman filter papers in a 55 C oven before the pre-hybridization, hybridization and autoradiography steps for the 3′ external probe as described above.

### Example 10: Generation of Targeted Transgenes at STMN2 Locus in hESC Via CRISPR/Cas9

STMN2 is a neuron-specific gene, which allows for relatively neuron-specific expression of the targeted transgene from the STMN2 locus. Site-specific genome editing via CRISPR/Cas9 was used to insert sequences coding for SNCA into endogenous genes.

To target the SNCA-GFP cassette into the STMN2 locus, a plasmid was generated bearing ˜1800 bp of homology surrounding the STMN2 stop codon. An IRES-SNCA-GFP coding sequence was then cloned into the STMN2 homologous sequence such that ˜900 bp of homology flanked the IRES-SNCA-GFP cassette. A FRT flanked PGK-Neomycin cassette was then cloned between the IRES-SNCA-GFP cassette and the STMN2 3′ homology arm. To incorporate the cassette into the STMN2 locus, 800,000 H9 hES cells were nucleofected using the Amaxa P3 Primary Cell 4D-Nucleofector X Kit with program CA137. The nucleofection reaction contained 15 μg of sgRNA (5′-tgtctggctgaagcaaggga-3′), 20 μg of ThermoFisher Truecut Cas9 v2 protein and 5.5 μg of the STMN2 targeting plasmid. After the nucleofection, cells were plated in a 1:1 mixture of StemFlex and MEF conditioned StemFlex with Rock inhibitor. The cells were allowed to recover for 48 hours before G418 selection was initiated. After visible colonies survived the selection, they were picked and plated into a 96-well plate. The expanded cells were replica-plated into two 96-well plates, one of which was used for genotyping. PCR was used to confirm the proper integration of the 5′ (primers STMN2.FOR2 and IRES-REV) and 3′ (primers NEO-F and STMN2-REV1) arms of the targeting cassette into the STMN2 locus. After targeting confirmation, a clone was expanded and a CAG-FLPo-Puro cassette was nucleofected into the cells following the above protocol. Puromycin selection allowed for the identification of cells which expressed FLP recombinase and colonies derived from these cells were picked, expanded, and genotyped by PCR to confirm removal of the PGK-Neo cassette.

### Results

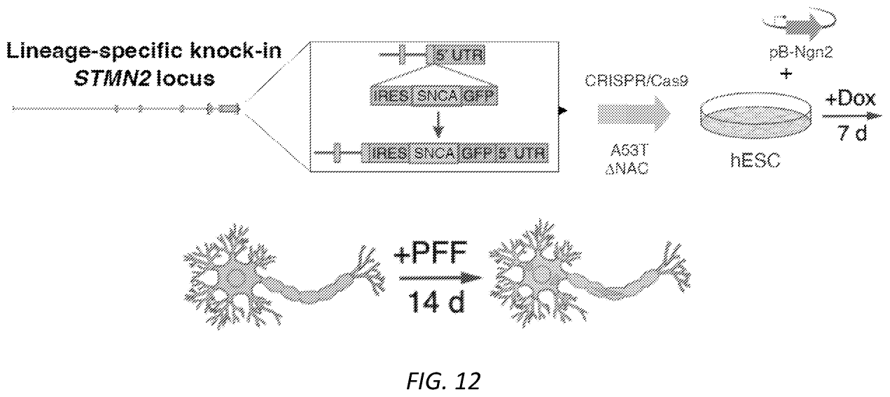

A construct with internal ribosome entry site (IRES) sequence followed by SNCA-GFP (IRES-SNCA-GFP) flanked by STMN2 homology arms (FIG. 12) was targeted to the STMN2 locus in H9 hESC by CRISPR/Cas9; insertion was confirmed by PCR. Knock-in of SNCA transgene at STMN2 locus did not lead to reduction in STMN2 expression (FIG. 13). Four cell lines were identified that had the intended insertion. One advantage of this system is that SNCA expression was under control of the neuron-specific STMN2 promoter, rendering the system doxycycline-independent. Doxycycline was only required to induce pB-Ngn2 expression.

### REFERENCES

1. Jeremic D, Jimenez-Diaz L, Navarro-Lopez J D. Past, present and future of therapeutic strategies against amyloid-beta peptides in Alzheimer's disease a systematic review. Ageing Res Rev. 2021:101496.
2. Rademakers R, Cruts M, van Broeckhoven C. The role of tau (MAPT) in frontotemporal dementia and related tauopathies. Hum Mutat. 2004; 24 (4): 277-95.
3. Diao J, Burre J, Vivona S, Cipriano D J, Sharma M, Kyoung M, et al. Native alpha-synuclein induces clustering of synaptic-vesicle mimics via binding to phospholipids and synaptobrevin-2/VAMP2. Elife. 2013; 2: e00592.
4. Sevigny J, Suhy J, Chiao P, Chen T, Klein G, Purcell D, et al. Amyloid PET Screening for Enrichment of Early-Stage Alzheimer Disease Clinical Trials: Experience in a Phase 1b Clinical Trial. Alzheimer Dis Assoc Disord. 2016; 30 (1): 1-7.
5. Jarosz D F, Khurana V. Specification of Physiologic and Disease States by Distinct Proteins and Protein Conformations. Cell. 2017; 171 (5): 1001-14.
6. Canals I, Ginisty A, Quist E, Timmerman R, Fritze J, Miskinyte G, et al. Rapid and efficient induction of functional astrocytes from human pluripotent stem cells. Nat Methods. 2018; 15 (9): 693-6.
7. Ng A H M, Khoshakhlagh P, Rojo Arias J E, Pasquini G, Wang K, Swiersy A, et al. A comprehensive library of human transcription factors for cell fate engineering. Nat Biotechnol. 2021; 39 (4): 510-9.
8. Chung C Y, Khurana V, Auluck P K, Tardiff D F, Mazzulli J R, Soldner F, et al. Identification and rescue of alpha-synuclein toxicity in Parkinson patient-derived neurons. Science. 2013; 342 (6161): 983-7.
9. Shahmoradian S H, Lewis A J, Genoud C, Hench J, Moors T E, Navarro P P, et al. Lewy pathology in Parkinson's disease consists of crowded organelles and lipid membranes. Nat Neurosci. 2019; 22 (7): 1099-109.
10. Goedert M, Spillantini M G, Del Tredici K, Braak H. 100 years of Lewy pathology. Nat Rev Neurol. 2013; 9 (1): 13-24.
11. Twohig D, Nielsen H M. alpha-synuclein in the pathophysiology of Alzheimer's disease. Mol Neurodegener. 2019; 14 (1): 23.
12. Peelaerts W, Bousset L, Van der Perren A, Moskalyuk A, Pulizzi R, Giugliano M, et al. alpha-Synuclein strains cause distinct synucleinopathies after local and systemic administration. Nature. 2015; 522 (7556): 340-4.
13. Guo J L, Covell D J, Daniels J P, Iba M, Stieber A, Zhang B, et al. Distinct alpha-synuclein strains differentially promote tau inclusions in neurons. Cell. 2013; 154 (1): 103-17.
14. Prusiner S B, Woerman A L, Mordes D A, Watts J C, Rampersaud R, Berry D B, et al. Evidence for alpha-synuclein prions causing multiple system atrophy in humans with parkinsonism. Proc Natl Acad Sci USA. 2015; 112 (38): E5308-17.
15. Peng C, Gathagan R J, Covell D J, Medellin C, Stieber A, Robinson J L, et al. Cellular milieu imparts distinct pathological alpha-synuclein strains in alpha-synucleinopathies. Nature. 2018; 557 (7706): 558-63.
16. Khurana V, Peng J, Chung C Y, Auluck P K, Fanning S, Tardiff D F, et al. Genome-Scale Networks Link Neurodegenerative Disease Genes to alpha-Synuclein through Specific Molecular Pathways. Cell Syst. 2017; 4 (2): 157-70 e14.
17. Kanazawa T, Uchihara T, Takahashi A, Nakamura A, Orimo S, Mizusawa H. Three-layered structure shared between Lewy bodies and lewy neurites-three-dimensional reconstruction of triple-labeled sections. Brain Pathol. 2008; 18 (3): 415-22.
18. Irwin D J, White M T, Toledo J B, Xie S X, Robinson J L, Van Deerlin V, et al. Neuropathologic substrates of Parkinson disease dementia. Ann Neurol. 2012; 72 (4): 587-98.
19. Shulman J M, De Jager P L, Feany M B. Parkinson's disease: genetics and pathogenesis. Annu Rev Pathol. 2011; 6:193-222.
20. Dettmer U, Ramalingam N, von Saucken V E, Kim T E, Newman A J, Terry-Kantor E, et al. Loss of native alpha-synuclein multimerization by strategically mutating its amphipathic helix causes abnormal vesicle interactions in neuronal cells. Hum Mol Genet. 2017; 26 (18): 3466-81.
21. Nuber S, Rajsombath M, Minakaki G, Winkler J, Muller C P, Ericsson M, et al. Abrogating Native alpha-Synuclein Tetramers in Mice Causes a L-DOPA-Responsive Motor Syndrome Closely Resembling Parkinson's Disease. Neuron. 2018; 100 (1): 75-90 e5.
22. Woodard L E, Wilson M H. piggyBac-ing models and new therapeutic strategies. Trends Biotechnol. 2015; 33 (9): 525-33.
23. Khurana V, Tardiff D F, Chung C Y, Lindquist S. Toward stem cell-based phenotypic screens for neurodegenerative diseases. Nat Rev Neurol. 2015; 11 (6): 339-50.
24. Hockemeyer D, Soldner F, Beard C, Gao Q, Mitalipova M, DeKelver R C, et al. Efficient targeting of expressed and silent genes in human ESCs and iPSCs using zinc-finger nucleases. Nat Biotechnol. 2009; 27 (9): 851-7.
25. Thomson J A, Itskovitz-Eldor J, Shapiro S S, Waknitz M A, Swiergiel J J, Marshall V S, et al. Embryonic stem cell lines derived from human blastocysts. Science. 1998; 282 (5391): 1145-7.
26 Takahashi K, Tanabe K, Ohnuki M, Narita M, Ichisaka T, Tomoda K, et al. Induction of pluripotent stem cells from adult human fibroblasts by defined factors. Cell. 2007; 131 (5): 861-72.
27. Zhang Y, Pak C, Han Y, Ahlenius H, Zhang Z, Chanda S, et al. Rapid single-step induction of functional neurons from human pluripotent stem cells. Neuron. 2013; 78 (5): 785-98.
28. Li S, Zhang A, Xue H, Li D, Liu Y. One-Step piggyBac Transposon-Based CRISPR/Cas9 Activation of Multiple Genes. Mol Ther Nucleic Acids. 2017; 8:64-76.
29 Tchieu J, Calder E L, Guttikonda S R, Gutzwiller E M, Aromolaran K A, Steinbeck J A, et al. NFIA is a gliogenic switch enabling rapid derivation of functional human astrocytes from pluripotent stem cells. Nat Biotechnol. 2019; 37 (3): 267-75.
30. Arenas E, Denham M, Villaescusa J C. How to make a midbrain dopaminergic neuron. Development. 2015; 142 (11): 1918-36.
31. Tipanee J, VandenDriessche T, Chuah M K. Transposons: Moving Forward from Preclinical Studies to Clinical Trials. Hum Gene Ther. 2017; 28 (11): 1087-104.
32. Zhao S, Jiang E, Chen S, Gu Y, Shangguan A J, Lv T, et al. PiggyBac transposon vectors: the tools of the human gene encoding. Transl Lung Cancer Res. 2016; 5 (1): 120-5.
33. Ausubel F M. Current Protocols in Molecular Biology: Greene Publishing Associates; 1989.
34. Vancura A. Transcriptional Regulation: Methods and Protocols Humana Press; 2011.
35. Stadtfeld M, Hochedlinger K. Without a trace? PiggyBac-ing toward pluripotency. Nat Methods. 2009; 6 (5): 329-30.
36. Grabundzija I, Irgang M, Mates L, Belay E, Matrai J, Gogol-Doring A, et al. Comparative analysis of transposable element vector systems in human cells. Mol Ther. 2010; 18 (6): 1200-9.
37. Dafnis I, Argyri L, Chroni A. Amyloid-peptide beta 42 Enhances the Oligomerization and Neurotoxicity of apoE4: The C-terminal Residues Leu279, Lys282 and Gln284 Modulate the Structural and Functional Properties of apoE4. Neuroscience. 2018; 394:144-55.
38. Weggen S, Beher D. Molecular consequences of amyloid precursor protein and presenilin mutations causing autosomal-dominant Alzheimer's disease. Alzheimers Res Ther. 2012; 4 (2): 9.
39. Tacik P, Sanchez-Contreras M, Rademakers R, Dickson D W, Wszolek Z K. Genetic Disorders with Tau Pathology: A Review of the Literature and Report of Two Patients with Tauopathy and Positive Family Histories. Neurodegener Dis. 2016; 16 (1-2): 12-21.
40. Lanoiselee H M, Nicolas G, Wallon D, Rovelet-Lecrux A, Lacour M, Rousseau S, et al. APP, PSEN1, and PSEN2 mutations in early-onset Alzheimer disease: A genetic screening study of familial and sporadic cases. PLOS Med. 2017; 14 (3): e1002270.
41. Goedert M, Jakes R. Mutations causing neurodegenerative tauopathies. Biochim Biophys Acta. 2005; 1739 (2-3): 240-50.
42. Caroppo P, Camuzat A, Guillot-Noel L, Thomas-Anterion C, Couratier P, Wong T H, et al. Defining the spectrum of frontotemporal dementias associated with TARDBP mutations. Neurol Genet. 2016; 2 (3): e80.
43. Yang C, Wang H, Qiao T, Yang B, Aliaga L, Qiu L, et al. Partial loss of TDP-43 function causes phenotypes of amyotrophic lateral sclerosis. Proc Natl Acad Sci USA. 2014; 111 (12): E1121-9.
44. Arnold E S, Ling S C, Huelga S C, Lagier-Tourenne C, Polymenidou M, Ditsworth D, et al. ALS-linked TDP-43 mutations produce aberrant RNA splicing and adult-onset motor neuron disease without aggregation or loss of nuclear TDP-43. Proc Natl Acad Sci USA. 2013; 110 (8): E736-45.
45. Meyer-Luehmann M, Coomaraswamy J, Bolmont T, Kaeser S, Schaefer C, Kilger E, et al. Exogenous induction of cerebral beta-amyloidogenesis is governed by agent and host. Science. 2006; 313 (5794): 1781-4.
46. Stopschinski B E, Diamond M I. The prion model for progression and diversity of neurodegenerative diseases. Lancet Neurol. 2017; 16 (4): 323-32.
47. Soper J H, Roy S, Stieber A, Lee E, Wilson R B, Trojanowski J Q, et al. Alpha-synuclein-induced aggregation of cytoplasmic vesicles in Saccharomyces cerevisiae. Mol Biol Cell. 2008; 19 (3): 1093-103.
48. Luk K C, Kehm V, Carroll J, Zhang B, O'Brien P, Trojanowski J Q, et al. Pathological alpha-synuclein transmission initiates Parkinson-like neurodegeneration in nontransgenic mice. Science. 2012; 338 (6109): 949-53.
49. Wakabayashi K, Tanji K, Odagiri S, Miki Y, Mori F, Takahashi H. The Lewy body in Parkinson's disease and related neurodegenerative disorders. Mol Neurobiol. 2013; 47 (2): 495-508.
50. Volpicelli-Daley L A, Luk K C, Lee V M. Addition of exogenous alpha-synuclein preformed fibrils to primary neuronal cultures to seed recruitment of endogenous alpha-synuclein to Lewy body and Lewy neurite-like aggregates. Nat Protoc. 2014; 9 (9): 2135-46.
51. Obrecht D. V J M. Solid-Supported Combinatorial and Parallel Synthesis of Small-Molecular-Weight Compound Libraries. 1st ed. Oxford: Pergamon, Elsevier Science Limited; 1998 Sep. 1, 1998.
52. Czarnik A W. Encoding methods for combinatorial chemistry. Curr Opin Chem Biol. 1997; 1 (1): 60-6.
53. Bennett C F, Krainer A R, Cleveland D W. Antisense Oligonucleotide Therapies for Neurodegenerative Diseases. Annu Rev Neurosci. 2019; 42:385-406.
54. Leavitt B R, Tabrizi S J. Antisense oligonucleotides for neurodegeneration. Science. 2020; 367 (6485): 1428-9.
55. Katsinelos T, Tuck B J, Mukadam A S, McEwan W A. The Role of Antibodies and Their Receptors in Protection Against Ordered Protein Assembly in Neurodegeneration. Front Immunol. 2019; 10:1139.
56. Yu Y J, Watts R J. Developing therapeutic antibodies for neurodegenerative disease. Neurotherapeutics. 2013; 10 (3): 459-72.
57. Valera E, Spencer B, Masliah E. Immunotherapeutic Approaches Targeting Amyloid-beta, alpha-Synuclein, and Tau for the Treatment of Neurodegenerative Disorders. Neurotherapeutics. 2016; 13 (1): 179-89.
58. Griffiths A J F. Genomics. W. M. Gelbart J H M, R. C. Lewontin editor. New York: W. H. Freeman and Company; 1999.
59. Ekins R, Chu F W. Microarrays: their origins and applications. Trends Biotechnol. 1999; 17 (6): 217-8.
60. MacBeath G, Schreiber S L. Printing proteins as microarrays for high-throughput function determination. Science. 2000; 289 (5485): 1760-3.
61. Simpson R J. Proteins and Proteomics: A Laboratory Manual: Cold Spring Harbor Laboratory Press; 2002.
62. Hardiman G. Microarrays Methods and Applications: DNA Press; 2003 July 1st, 2003.
63. Ryan S D, Dolatabadi N, Chan S F, Zhang X, Akhtar M W, Parker J, et al. Isogenic human iPSC Parkinson's model shows nitrosative stress-induced dysfunction in MEF2-PGC1alpha transcription. Cell. 2013; 155 (6): 1351-64.
64. Soto C, Saborio G P, Anderes L. Cyclic amplification of protein misfolding: application to prion-related disorders and beyond. Trends Neurosci. 2002; 25 (8): 390-4.
65. Bargar C, Wang W, Gunzler S A, LeFevre A, Wang Z, Lerner A J, et al. Streamlined alpha-synuclein RT-QuIC assay for various biospecimens in Parkinson's disease and dementia with Lewy bodies. Acta Neuropathol Commun. 2021; 9 (1): 62.
66. Richardson C D, Ray G J, DeWitt M A, Curie G L, Corn J E. Enhancing homology-directed genome editing by catalytically active and inactive CRISPR-Cas9 using asymmetric donor DNA. Nat Biotechnol. 2016; 34 (3): 339-44.
67. Dettmer U, Newman A J, von Saucken V E, Bartels T, Selkoe D. KTKEGV repeat motifs are key mediators of normal alpha-synuclein tetramerization: Their mutation causes excess monomers and neurotoxicity. Proc Natl Acad Sci USA. 2015; 112 (31): 9596-601.
68. Wang, T., Birsoy, K., Hughes, N. W., Krupczak, K. M., Post, Y., Wei, J. J., Lander, E. S., and Sabatini, D. M. (2015). Identification and characterization of essential genes in the human genome. Science 350, 1096-1101. 10.1126/science.aac7041.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A PiggyBac vector comprising:
a sequence encoding a target protein selected from the group consisting of TAR DNA-binding protein (TARDBP, TDP-43), apolipoprotein E (ApoE), α-synuclein (SNCA), beta-amyloid, amyloid precursor protein (APP), and tau, wherein the target protein causes formation of proteotoxic or proteinaceous inclusions in the cell;
at least one pair of insulators;
at least one antibiotic selection gene;
an inducible promoter, optionally a tet-inducible promoter;
optionally, a neuronal differentiation transcription factor, preferably Ngn2, NFIB, or Sox9; and
a herpes simplex virus thymidine kinase selection gene.

2. The vector of claim 1, wherein the target protein is α-syn or TARDBP.

3. The vector of claim 1, wherein the sequence encoding the target protein encodes an amino acid sequence comprising a wild type version of the target protein, or an amino acid sequence containing a disease risk-associated polymorphism or mutation.

4. The vector of claim 2, wherein the alpha-synuclein comprises E35K, E46K, and/or E61K point mutations, or the TARDBP comprises Q331K or M337V point mutations.

5. The vector of claim 1, wherein a green fluorescent protein (GFP) is linked to the target protein.

6. A method of generating a human transgenic cellular model of neurodegenerative proteinopathies comprising:
transducing a human cell with a PiggyBac vector, wherein the PiggyBac vector comprises a sequence encoding a target protein selected from the from the group consisting of apolipoprotein E (ApoE), TARDBP, α-syn, beta-amyloid, amyloid precursor protein (APP), and tau, wherein expression of the target protein causes proteotoxic or proteinaceous inclusions in the cell, wherein the PiggyBac vector comprises at least one pair of insulators, at least one antibiotic selection gene, and a herpes simplex virus thymidine kinase selection gene.

7. The method of claim 6, wherein the target protein is α-syn or TARDBP.

8. The method of claim 6, wherein the sequence encoding the target protein encodes an amino acid sequence comprising a wild type version of the target protein, or an amino acid sequence containing a disease risk-associated polymorphism or mutation.

9. The method of claim 6, wherein the PiggyBac vector comprises an inducible promoter, preferably a tet-inducible promoter.

10. The method of claim 6, wherein the PiggyBac vector comprises 2 or 4 insulators, preferably UCOE insulator, iA4 insulator, cHS4 insulator, or iA2 insulator.

11. The method of claim 6, wherein a green fluorescent protein (GFP) is linked to the target protein.

12. The method of claim 7, wherein the α-syn comprises E35K, E46K, and E61K point mutations, or the TARDBP comprises Q331K or M337V point mutations.

13. The method of claim 6, wherein the human cell is selected from the group consisting of a pluripotent stem cell (iPSC), an embryonic stem cell (ESc), and a cell from an immortalized cell line.

14. The method of claim 13, wherein the human cell is the iPSC.

15. The method of claim 13, wherein the PiggyBac vector further comprises a neuronal differentiation transcription factor, preferably Ngn2, NFIB, or Sox9, coding sequence.

16. The method of claim 15, wherein the iPSC is differentiated to a cortical neuron cell by expression of Ngn2; to an astrocyte by expression of NFIB; or an oligodendrocyte by expression of Sox9.

17. The method of claim 14, wherein the iPSC comprises a disease risk-associated polymorphism or mutation in a gene selected from a group comprising α-syn, TARDBP, APP, tau or ApoE.

18. A method comprising generating a human cell comprising a target gene, wherein the target gene is introduced into the genome of the cell by CRISPR, encoding a target protein selected from the group consisting of apolipoprotein E (ApoE), TARDBP, α-syn, beta-amyloid, amyloid precursor protein (APP), and tau, wherein the target protein causes formation of proteotoxic or proteinaceous inclusions in the cell and is introduced into the AAVS1 locus or STMN2 locus.

19. The method of claim 18, wherein the target gene is introduced into the STMN2 locus.

20. The method of claim 18, further comprising introducing a neuronal differentiation transcription factor (e.g., Ngn2, NFIB, or Sox9) gene and a tet-inducible promoter.

21. The method of claim 18, wherein the target protein comprises an amino acid sequence containing a disease risk-associated polymorphism or mutation.

22. The method of claim 18, wherein the target protein is α-syn or TARDBP.

23. The method of claim 22, wherein the α-syn comprises E35K, E46K, and E61K point mutations, or the TARDBP comprises Q331K or M337V point mutations.

24. The method of claim 18, wherein a fluorescent protein, optionally a green fluorescent protein (GFP), is linked to the target protein.

25. The method of claim 18, wherein the human cell is selected from the group consisting of pluripotent stem cell (iPSc), an embryonic stem cell (ESc), and a cell from an immortalized cell line, preferably, an U2OS cell.

26. An isolated human cell comprising a PiggyBac vector, wherein the PiggyBac vector comprises a sequence encoding a target protein selected from the group consisting of apolipoprotein E (ApoE), α-syn, beta-amyloid, amyloid precursor protein (APP), and tau, wherein expression of the target protein causes proteotoxic or proteinaceous inclusions in the cell, wherein the PiggyBac vector comprises at least one pair of insulators, at least one antibiotic selection gene, and a herpes simplex virus thymidine kinase selection gene.

27. The isolated human cell of claim 26, wherein the human cell is selected from the group consisting of pluripotent stem cell (iPSc), an embryonic stem cell (ESc), and a cell from an immortalized cell line, preferably an U2OS cell.

28. The isolated human cell of claim 26, wherein the human cell is differentiated into neurons or glial cells, preferably cortical neurons, dopaminergic neurons, astrocytes, oligodendrocytes, microglia.

29. An isolated human cell comprising an apolipoprotein E (ApoE), α-syn, TARDBP, beta-amyloid, amyloid precursor protein (APP), or tau gene expressing from an AAVS1 locus and an Ngn2 gene, wherein the cell is generated by:
contacting a human cell, preferably an hESc or iPSC cell, with an RNA-guided Cas9 nuclease and gRNA directed to a sequence in the AAVS1 locus, and a sequence under an inducible promoter encoding ApoE, TARDBP, α-syn, beta-amyloid, APP, or tau, under conditions allowing insertion of the ApoE, TARDBP, α-syn, beta-amyloid, APP, or tau gene into the AAVS1 locus;
differentiating the human cell into a neuron or glial cells by expressing a neuronal differentiation transcription factor, preferably Ngn2, NFIB, or Sox9; and
maintaining the cell under conditions suitable for expression of ApoE, TARDBP, α-syn, beta-amyloid, APP, or tau to cause proteotoxic or proteinaceous inclusions in the cell.

30. An isolated human cell comprising (i) a sequence encoding apolipoprotein E (ApoE), TARDBP, α-syn, beta-amyloid, amyloid precursor protein (APP), or tau protein inserted in a STMN2 or AAVS1 locus, and (ii) an exogenous neuronal differentiation transcription factor, preferably Ngn2, NFIB, or Sox9gene, wherein the cell is generated by:
contacting the human cell, preferably an hESc or iPSC cell, with an RNA-guided Cas9 nuclease and gRNA directed to a sequence in the AAVS1 or STMN2 locus, and a sequence encoding TARDBP, ApoE, alpha-synuclein, beta-amyloid, APP, or tau, under conditions allowing insertion of the TARDBP, ApoE, α-syn, beta-amyloid, APP, or tau gene into the AAVS1 or STMN2 locus;
differentiating the human cell into a neuron or glial cells by expressing the neuronal differentiation transcription factor; and
optionally maintaining the differentiated cells under conditions suitable for expression of TARDBP, ApoE, α-syn, beta-amyloid, App, or tau to cause proteotoxic or proteinaceous inclusions in the cell.

31. A method of identifying a candidate compound for treating a neurodegenerative condition associated with proteotoxic or proteinaceous inclusions in neuronal or glial cells, the method comprising:
contacting the human cell of claims 26-30 with a test compound, optionally in the presence and absence of fibrils;
evaluating a level of proteotoxic or proteinaceous inclusions in the human cell in the presence and absence of the candidate compound; and
selecting as a candidate compound a test compound that reduces the level of proteotoxic or proteinaceous inclusions in the human cell in the presence of fibrils.

32. A method of identifying a candidate gene therapy for treating a neurodegenerative condition associated with proteotoxic or proteinaceous inclusions in neuronal or glial cells, the method comprising:
contacting the human cell of claims 26-30 with a vector comprising a single gene or library of genes that over-express, knockdown or knock-out one or more genes in the human genome;
evaluating a level of proteotoxic or proteinaceous inclusions in the human cell in the presence and absence of the candidate compared; and
selecting as a candidate gene a specific gene target or combination or targets that reduces the level of proteotoxic or proteinaceous inclusions in the human cell.
